(19) United States
(12) Patent Application Publication
(10) Pub. No.: US 2023/0201800 A1
(43) Pub. Date: Jun. 29, 2023

(54) AIR PERMEABLE FILTER MATERIAL COMPRISING A POLYMER AEROGEL

(71) Applicant: Blueshift Materials, Inc., Spencer, MA (US)

(72) Inventors: Marisa SNAPP-LEO, Spence, MA (US); Lawino KAGUMBA, Spencer, MA (US); Garrett POE, Spencer, MA (US); Timothy BURBEY, Spencer, MA (US)

(73) Assignee: Blueshift Materials, Inc., Spencer, MA (US)

(21) Appl. No.: 17/996,817

(22) PCT Filed: Apr. 23, 2021
(86) PCT No.: PCT/US2021/028948

§ 371 Date: Oct. 21, 2022

Related U.S. Application Data
(60) Provisional application No. 63/025,527, filed on May 15, 2020.
(60) Provisional application No. 63/015,337, filed on Apr. 24, 2020.

(51) Int. Cl. B01J 20/26; C08G 73/10; C08J 9/28; B01J 20/28; B01J 20/30; A61L 9/014; A61L 2/18; A61L 2/22; A62B 23/02; A62B 18/08; B01D 39/16; B01D 39/04; A41D 13/11; A41D 31/14

(52) U.S. Cl. CPC B01J 20/262; C08G 73/1042; C08G 73/1071; C08G 73/1032; C08J 9/286; B01J 20/28047; B01J 20/2805; B01J 20/28035; B01J 20/28028; B01J 20/28083; B01J 20/28085; B01J 20/3085; B01J 20/3064; A61L 9/014; A61L 2/18; A61L 2/22; A62B 23/025; A62B 18/084; A62B 23/02; B01D 39/1676; B01D 39/04; A41D 13/11; A41D 31/14; A61L 2209/14; A61L 2209/22; C08J 2201/0502; C08J 2205/026; C08J 2205/05; C08J 2205/044; C08J 2379/08; B01D 2239/0442; B01D 2239/1216; B01D 2239/10; B01D 2239/0654

(57) ABSTRACT

An air-permeable filter material that includes a polymeric aerogel having a polymeric matrix comprising an open-cell structure is disclosed. The air-permeable filter material can be included in a mask, which can be configured to be placed over a user's mouth and/or nose. The mask can include at least one layer of the air-permeable filter material and is positioned such that inhaled and/or exhaled air of the user passes through the filter material.

### CROSS-REFERENCE TO RELATED APPLICATIONS

This application claims the benefit to U.S. Provisional Application No. 63/025,527, filed May 15, 2020, and to U.S. Provisional Application 63/015,337, filed Apr. 24, 2020. The contents of the referenced applications are incorporated into the present application by reference.

### BACKGROUND OF THE INVENTION

A. Field of the Invention

The present disclosure relates to the field of air permeable filters. In the context of the present invention, the air permeable filers can include an air permeable polymeric aerogel layer or film having a polymeric matrix. The filter material can be used in personal protection equipment (PPE) such as face masks.

B. Description of Related Art

PPE in the form of face masks (e.g., surgical face masks, painters face masks, etc.) are typically used by health care professionals and others to protect the user from inhaling and expelling harmful materials and particles such as airborne pathogens. For example, masks can limit airborne pathogens such as bacterial or viral particles from entering into a person's nasal cavity or mouth. The masks can also limit the shedding of bacterial or viral particles onto other surfaces.

One of the issues with currently available PPE (e.g., N95 qualified facial masks and respirator cartridge filters) is that the filtration materials in the masks cannot withstand the cleaning, sterilization, and/or disinfection process, and thus cannot safely be reused. One of the issues with currently available facial masks (e.g., N95 qualified facial masks) is that they cannot safely be reused without having to wash or disinfect the masks. As seen in today's society with the novel coronavirus (SARS-CoV-2) pandemic, this can lead to a diminishing supply of PPE masks. This can, in turn, lead to increased infections.

### SUMMARY OF THE INVENTION

A discovery has been made that provides a solution to at least some of the problems associated with PPE and/or other equipment that uses filters to filter out undesired particles from air. In one aspect, the solution resides in the use of polymeric aerogels that have a polymeric matrix with an open-cell structure in filtration applications. The polymeric aerogels can be organic polymeric aerogels (e.g., polyimide aerogels). The aerogels can be in the form of a film or layer, a multiple films or layers, and can be used as a breathable filter material that is capable of entrapping undesired particles (e.g., airborne pathogens such as virus or bacterial particles) while allowing air to flow through. In another embodiment, the aerogels can be in particulate form, and the particles can be used to form a filter medium by placing the particles in, for example, a pouch, cartridge, or support material. The breathable filter material can be incorporated into PPE equipment (e.g., face or surgical masks, or cartridge-type respirators) and be used as filter medium for such equipment. Notably, the polymeric aerogels used to make the filter material have a sufficient amount of structural integrity and durability to commonly-used disinfectant, cleaning, and sterilization agents that allows the material to be washed or disinfected or sterilized and reused. This provides an advantage in that it can reduce or prevent PPE shortages such as the shortages seen in today's society, which is dealing with the novel coronavirus (SARS-CoV-2) pandemic. Further, the porous structure of the polymeric aerogels allows for a sufficient amount of air to flow through the aerogel while preventing or limited the flow of viral or bacterial particles, such as, for example particles of SARS-CoV-2. In particular embodiments, the average pore size of the aerogel filter material can be 50 nanometers (nm) to 500 nm, preferably 100 nm to 400 nm, more preferably 200 nm to 300 nm, or even more preferably about 225 nm to about 275 nm, or about 250 nm. An advantage of these pore sizes coupled with the open-cell structure of the polymeric matrix allows for a sufficient amount of air to flow through such that the filter is breathable but reduces or prevents undesired materials such as viral or bacterial particles from flowing through. As discussed below, additional pore sizes below and above these ranges are also contemplated in the context of the present invention. With that said, pore sizes of less than 50 nm can reduce breathability and/or can also reduce the amount of airborne pathogens that can be filtered out as the surface of the material can quickly become saturated particles of the airborne pathogens, which can lead to having to increase washing, disinfecting, or sterilization cycles. Pore sizes of greater than 500 nm can lead to greater breathability, but can also lead to reduced effectiveness of the material to filter out airborne pathogens.

In one aspect of the present invention, there is disclosed an air-permeable filter material that comprises a polymeric aerogel having a polymeric matrix. The matrix can have an open-cell structure such that the pores can be fluidly connected such that air can pass through the matrix. In one aspect, the air-permeable filter material can be included in a mask or cartridge filter for a cartridge-type respirator. The mask can be configured to be placed over a user's mouth and/or nose. The mask can include at least one layer of the air-permeable filter material and can be positioned such that inhaled and/or exhaled air of the user passes through the filter material. The mask can include at least one strap that is attached to at least one or two different parts of the mask (e.g., a first side and a second side of the mask). A portion of the strap can be capable of being positioned behind a user's head. The at least one strap can include a stretchable or elastic portion. In another aspect, the air-permeable filter material can be comprised in a vent filter. In some aspects of the present invention, the polymeric aerogel can be in the form of a film or can be in particulate form or a combination thereof. In one embodiment, the air-permeable filter material can include a polymeric aerogel film(s), and the film(s) can be positioned to form a pouch or internal space. The pouch or internal space can be loaded with particles of polymeric aerogel to provide a dual or enhanced air-filtration effect. The vent filter can have a first surface and an opposing second surface. The shape of the first and second surfaces can be square or rectangular in shape and can be capable of fitting into a wall or ceiling or floor vent. The vent filter can include an outer border. The outer border can include a paper material (e.g., cardboard or cotton). In other aspects, the air-permeable filter material can be attached to a substrate. The substrate can include a paper material, a fibrous material, a metal material (e.g., a metal mesh or screen), a thermoplastic material, or a thermoset material. In some aspects, the substrate is a fiber substrate, preferably, a woven fiber substrate, a knitted fiber substrate, non-woven fiber substrate, or a paper substrate. In some aspects, the air-permeable filter material is a N95, N99, or N100 qualified filter material. In another aspect, the air-permeable filter material is a R95, R99, and R100 qualified filter material. In another aspect, the air-permeable filter material is a P95, P99, or P100 qualified filter material. In another aspect, the air-permeable filter material is a high efficiency (HE) qualified filter material. The qualification can be the U.S. National Institute for Occupational Safety and Health (NIOSH) standard, which can include the following standard in Table 1:

TABLE 1

| Filter Class | Description |
|---|---|
| N95, N99, N100 | Filters at least 95%, 99%, 99.97% of airborne particles. |
| | Not resistant to oil. |
| R95, R99, R100 | Filters at least 95%, 99%, 99.97% of airborne particles. |
| | Somewhat resistant to oil. |
| P95, P99, P100 | Filters at least 95%, 99%, 99.97% of airborne particles. |
| | Strongly resistant to oil. |
| HE (High Efficiency | Filters at least 99.97% of airborne particles. |
| Particulate Air) | For use on PAPRs only. PAPRs use only HE filters. |

In another aspect, the air-permeable filter material can be capable of removing an airborne pathogen from air by passing air comprising the airborne pathogen through at least a portion of the filter material such that the airborne pathogen is retained in the filter material and air is allowed to pass through the filter material. The airborne pathogen can be a virus, a bacteria, a fungi, or a protozoa. The airborne pathogen can be a virus, and wherein the virus is preferably an adenovirus, alphavirus, calicivirus, coronavirus, distemper virus, Ebola virus, enterovirus, flavivirus, hepatitis virus, herpesvirus, infectious peritonitis virus, leukemia virus, Marburg virus, Norwalk virus, orthomyxovirus, papilloma virus, parainfluenza virus., the, paramyxovirus, parvovirus, pestivirus, picorna virus, pox virus, rabies virus, reovirus polypeptides, retrovirus, rotavirus, and vaccinia virus. The airborne pathogen can be a coronavirus. The coronavirus can be MERS-CoV, SARS-CoV, or SARS-CoV-2, preferably SARS-CoV-2. The airborne pathogen can be a bacteria, and wherein the bacterial is preferably Actinomyces, Bacillus, Bacteroides, Bordetella, Bartonella, Borrelia, Brucella, Campylobacter, Capnocytophaga, Chlamydia, Corynebacterium, Coxiella, Dermatophilus, Enterococcus, Ehrlichia, Escherichia, Francisella, Fusobacterium, Haemobartonella, Haemophilus, Helicobacter, Klebsiella, L-form bacteria, Leptospira, Listeria, Mycobacteria, Mycoplasma, Neisseria, Neorickettsia, Nocardia, Pasteurella, Peptococcus, Peptostreptococcus, Pneumococcus, Proteus, Pseudomonas, Rickettsia, Rochalimaea polypeptides, Salmonella, Shigella, Staphylococcus, group A streptococcus, group B streptococcus, Treponema, and Yersinia. The airborne pathogen is a fungi, and wherein the fungi is preferably Absidia, Acremonium, Alternaria, Aspergillus, Basidiobolus, Bipolaris, Blastomyces, Candida, Coccidioides, Conidiobolus, Cryptococcus, Curvalaria, Epidermophyton, Exophiala, Geotrichum, Histoplasma, Madurella, Malassezia, Microsporum, Moniliella, Mortierella, Mucor, Paecilomyces, Penicillium, Phialemonium, Phialophora, Prototheca, Pseudallescheria, Pseudomicrodochium, Pythium, Rhinosporidium, Rhizopus, Scolecobasidium, Sporothrix, Stemphylium, Trichophyton, Trichosporon, and Xylohypha. The airborne pathogen can be a protozoa, and wherein the protozoa is preferably Babesia, Balantidium, Besnoitia, Cryptosporidium, Eimeria, Encephalitozoon, Entamoeba, Giardia, Hammondia, Hepatozoon, Isospora, Leishmania, Microsporidia, Neospora, Nosema, Pentatrichomonas, Plasmodium. Examples of helminth parasites include, but are not limited to, Acanthocheilonema, Aelurostrongylus, Ancylostoma, Angiostrongylus, Ascaris, Brugia, Bunostomum, Capillaria, Chabertia, Cooperia, Crenosoma, Dictyocaulus, Dioctophyme, Dipetalonema, Diphyllobothrium, Diplydium, Dirofilaria, Dracunculus, Enterobius, Filaroides, Haemonchus, Lagochilascaris, Loa, Mansonella, Muellerius, Nanophyetus, Necator, Nematodirus, Oesophagostomum, Onchocerca, Opisthorchis, Ostertagia, Parafilaria, Paragonimus, Parascaris, Physaloptera, Protostrongylus, Setaria, Spirocerca Spirometra, Stephanofilaria, Strongyloides, Strongylus, Thelazia, Toxascaris, Toxocara, Trichinella, Trichostrongylus, Trichuris, Uncinaria, Wuchereria, Pneumocystis, Sarcocystis, Schistosoma, Theileria, Toxoplasma, and Trypanosoma. In still other embodiments, the polymeric aerogel can include micropores (pore size of less than 2 nanometers (nm), mesopores (pore size of 2 nm to 50 nm), or macropores (pore size greater than 50 nm), or a combination thereof. In one aspect, at least 10%, 50%, 75%, 95%, or 100% of the aerogel's pore volume is made up of micropores. In one aspect, at least 10%, 50%, 75%, 95%, or 100% of the aerogel's pore volume is made up of mesopores. In one aspect, at least 10%, 50%, 75%, 95%, or 100% of the aerogel's pore volume is made up of macropores. In one aspect, less than 90%, 80%, 70%, 60%, 50%, 40%, 30% 20%, 10% or less than 5% of the aerogel's pore volume is made up of micropores and/or mesopores. In one aspect, the polymeric matrix has an average pore size of 2 nanometers (nm) to 50 nm in diameter. In one aspect, the polymeric matrix has an average pore size of greater than 50 nanometers (nm) to 5000 nm in diameter. In one aspect, the polymeric matrix has an average pore size of 100 nm to 800 nm, preferably 100 nm to 500 nm, more preferably from 150 nm to 400 nm, even more preferably from 200 nm to 300 nm, still more preferably from 225 nm to 275 nm. In some aspects, the polymeric matrix has an average pore size of 1,000 nm to 1,400 nm, preferably 1,100 nm to 1,300 nm, or more preferably around 1,200 nm. The polymeric aerogel can be an organic polymeric aerogel (e.g. a polyimide aerogel or a polyamide aerogel) or a polymeric aerogel containing inorganic and organic compounds (e.g., polydimethylsiloxane aerogel). In some aspects, it could be an inorganic aerogel, such as one limited to inorganic compounds or one that can contain both inorganic and organic compounds (e.g., polydimethylsiloxane aerogel). In one aspect, the air permeable filter material can further comprise a support film or layer at least partially penetrating the polymer matrix of the aerogel. The support can be a fiber support. The fiber support can be a woven fiber support, knitted fiber support, non-woven fiber support or a paper. In one aspect, the fiber support can be a scrim that is adhesively bonded onto the air permeable filter material or the polymer aerogel. In one aspect, the air permeable filter material or the polymer aerogel or the combination thereof can be in the form of a film or layer having a thickness of 0.01 millimeters (mm) to 1000 mm thick, or from 0.025 mm to 100 mm thick, or from 0.050 mm to 10 mm thick, or from 0.100 mm to 2 mm thick, or from 0.125 mm to 1.5 mm.

Also disclosed in the context of the present invention is a method of removing airborne pathogens. The method can include passing air comprising the airborne pathogen through at least a portion of any one of the air permeable filter materials of the present invention such that the airborne pathogen is retained in the filter material and air is allowed to pass through the filter material. As noted above, the airborne pathogen can be a virus, a bacteria, a fungi, or a protozoa.

In another aspect, there is disclosed a method of sterilizing or disinfecting any one of the air permeable filter materials of the present invention. The method can include subjecting the material to a disinfectant or sterilization solution or spray capable of killing an airborne pathogen.

Also disclosed is a method of making any one of the air permeable filter materials of the present invention. The method can include (a) providing a monomer or a combination of monomers to a solvent to form a solution, (b) polymerizing the monomers in the solution to form a polymer gel matrix, and (c) subjecting the polymer gel matrix to conditions sufficient to remove liquid from the polymer gel matrix to form an aerogel having a polymeric matrix comprising an open-cell structure. Step (b) can further comprise adding a curing agent to the solution to reduce the solubility of polymers formed in the solution. The use of a curing agent can allow for the selective formation of micropores, mesopores, and/or macropores in the gel matrix, preferably macropores. The formed pores can contain liquid from the solution. The process can include casting the polymer gel matrix in step (b) onto a support such that a layer of the polymeric gel matrix is comprised on the support, wherein the aerogel in step (c) is in the form of a film. The process can also include crushing, grinding, or milling the aerogel film or stock or monolithic shapes of polymeric aerogel to produce aerogel particles. The aerogel particles can be any size. In some embodiments, the aerogel particle size can be 5 μm to 500 μm, or at least, equal to, or between any two of 5, 10 20, 30, 40, 50, 60, 70, 80, 90, 100, 150, 200, 250, 300, 350, 400, 450 and 500 μm. In some embodiments, the particle size distribution can be single-modal or multi-modal (e.g., bimodal, trimodal, etc.). In certain embodiments, the particle size distribution is bimodal with one mode being between 10 and 100 μm and the other mode being between 150 and 300 μm. The method can also comprise covering or immersing a support film in the solution in step (a) or (b) such that the support film is attached to the resulting polymer gel matrix and ultimately to the produced aerogel matrix. In one instance, and if macropores are desired to be present in the polymer aerogel matrix, then the formation of macropores versus smaller mesopores and micropores can be primarily controlled by controlling the polymer/solvent dynamics during gel formation. By doing so, the pore structure can be controlled, and the quantity and volume of macroporous, mesoporous, and microporous cells can be controlled. In one instance, this can be done by adding a curing agent to the solution to reduce the solubility of polymers formed in the solution and to form macropores in the gel matrix, the formed macropores containing liquid from the solution. For example, a curing additive (e.g., 1,4-diazabicyclo[2.2.2]octane) that can reduce the solubility of the polymers being formed during polymerization step (b), can produce a polymer gel containing a higher number of macropores compared to another curing additive (e.g., triethylamine) that improves the resultant polymer solubility. In another specific non-limiting example when forming a polyimide aerogel having macropores, increasing the ratio of rigid amines incorporated into the polymer backbone such as p-phenylenediamine (p-PDA) as compared to more flexible diamines such as 4,4′-oxydianiline (4,4′-ODA), the formation of macropores as compared to smaller mesopores and micropores can be controlled. More specifics about the monomers and solvents and processing conditions are provided below in the Detailed Description and Examples section of the present specification. In more general terms, the following processing variables/conditions can be used to favor the formation of macropores versus mesopores and/or micropores: (1) the polymerization solvent; (2) the polymerization temperature; (3) the polymer molecular weight; (4) the molecular weight distribution; (5) the copolymer composition; (6) the amount of branching; (7) the amount of crosslinking; (8) the method of branching; (9) the method of crosslinking); (10) the method used in formation of the gel; (11) the type of catalyst used to form the gel; (12) the chemical composition of the catalyst used to form the gel; (13) the amount of the catalyst used to form the gel; (14) the temperature of gel formation; (15) the type of gas flowing over the material during gel formation; (16) the rate of gas flowing over the material during gel formation; (17) the pressure of the atmosphere during gel formation; (18) the removal of dissolved gasses during gel formation; (19) the presence of solid additives in the resin during gel formation; (20) the amount of time of the gel formation process; (21) the substrate used for gel formation; (22) the type of solvent or solvents used in each step of the optional solvent exchange process; (23) the composition of solvent or solvents used in each step of the optional solvent exchange process; (24) the amount of time used in each step of the optional solvent exchange process; (25) the dwell time of the part in each step of the solvent exchange process; (26) the rate of flow of the optional solvent exchange solvent; (27) the type of flow of the optional solvent exchange solvent; (28) the agitation rate of the optional solvent exchange solvent; (29) the temperature used in each step of the optional solvent exchange process; (30) the ratio of the volume of optional solvent exchange solvent to the volume of the part; (31) the method of drying; (32) the temperature of each step in the drying process; (33) the pressure in each step of the drying process; (34) the composition of the gas used in each step of the drying process; (35) the rate of gas flow during each step of the drying process; (36) the temperature of the gas during each step of the drying process; (37) the temperature of the part during each step of the drying process; (38) the presence of an enclosure around the part during each step of the drying process; (39) the type of enclosure surrounding the part during drying; and/or (40) the solvents used in each step of the drying process.

Also disclosed in the context of the present invention are aspects 1 to 68. Aspect 1 is an air-permeable filter material that comprises a polymeric aerogel having a polymeric matrix comprising an open-cell structure. Aspect 2 is the air-permeable filter material of aspect 1, wherein the air-permeable filter material is comprised in a mask. Aspect 3 is the air-permeable filter material of aspect 2, wherein at least a portion of the mask is configured to be placed over a user's mouth and/or nose. Aspect 4 is the air-permeable filter material of any one of aspects 2 to 3, wherein the mask comprises at least one layer of the air-permeable filter material and is positioned such that inhaled and/or exhaled air of the user passes through the filter material. Aspect 5 is the air-permeable filter material of any one of aspects 2 to 4, wherein the mask comprises at least one strap that is attached to at least two different parts of the mask, and wherein at least a portion of the strap is capable of being positioned behind a user's head or ear. Aspect 6 is the air-permeable filter material of aspect 5, wherein the at least one strap comprises a stretchable or elastic portion. Aspect 7 is the air-permeable filter material of any one of aspects 1 to 6, wherein the air-permeable filter material is comprised in a cartridge. Aspect 8 is the air-permeable filter material of aspect 7, wherein the cartridge is a replaceable cartridge for a cartridge-type respirator. Aspect 9 is the air-permeable filter material of any one of aspects 7 to 8, wherein the air-permeable filter material is in the form of a film or is in the form of particles, or a combination thereof. Aspect 10 is the air-permeable filter material of aspect 9, wherein the cartridge has a space, and wherein the space is at least partially filled with the film or the particles or both of the film and particles. Aspect 11 is the air-permeable filter material of aspect 1, wherein the material is comprised in a vent filter. Aspect 12 is the air-permeable filter material of aspect 11, wherein the vent filter has a first surface and an opposing second surface, wherein the shape of the first and second surfaces are square or rectangular in shape. Aspect 13 is the air-permeable filter material of any one of aspects 11 to 12, wherein the vent filter comprises an outer border, wherein the outer border comprises a paper material. Aspect 14 is the air-permeable filter material of aspect 13, wherein the paper material is cardboard or cotton. Aspect 15 is the air-permeable filter material of any one of aspects 1 to 14, wherein the polymeric aerogel is in the form of a film or is in particulate form or a combination thereof. Aspect 16 is the air-permeable filter material of aspect 15, wherein the polymeric aerogel is in the form of a film. Aspect 17 is the air-permeable filter material of aspect 16, wherein the film is adhesively bonded to a support, preferably a scrim. Aspect 18 is the air-permeable filter material of aspect 15, wherein the polymeric aerogel is in particulate form. Aspect 19 is the air-permeable filter material of aspect 18, wherein the polymeric aerogel in particulate form is comprised in a support material, preferably a fibrous support material. Aspect 20 is the air-permeable filter material of any one of aspects 1 to 19, wherein the polymeric aerogel is attached to a substrate. Aspect 21 is the air-permeable filter material of aspect 20, wherein the substrate comprises a paper material, a fibrous material, a metal material, a thermoplastic material, or a thermoset material. Aspect 22 is the air-permeable filter material of aspect 21, wherein the substrate is a fiber substrate, preferably, a woven fiber substrate, a knitted fiber substrate, non-woven fiber substrate, or a paper substrate. Aspect 23 is the air-permeable filter material of any one of aspects 20 to 22, wherein the substrate is a scrim. Aspect 24 is the air-permeable filter material of any one of aspects 20 to 23, wherein the polymeric aerogel is attached to the substrate with an adhesive, preferably a polyester-based adhesive. Aspect 25 is the air-permeable filter material of any one of aspects 20 to 24, wherein the substrate is air permeable. Aspect 26 is the air-permeable filter material of any one of aspects 1 to 25, wherein the filter material is a N95, N99, or N100 qualified filter material. Aspect 27 is the air-permeable filter material of any one of aspects 1 to 25, wherein the filter material is a R95, R99, and R100 qualified filter material. Aspect 28 is the air-permeable filter material of any one of aspects 1 to 25, wherein the filter material is a P95, P99, or P100 qualified filter material. Aspect 29 is the air-permeable filter material of any one of aspects 1 to 25, wherein the filter material is a high efficiency (HE) qualified filter material. Aspect 30 is the air-permeable filter material of any one of aspects 1 to 29, wherein the filter material is capable of removing an airborne pathogen from air by passing air comprising the airborne pathogen through at least a portion of the filter material such that the airborne pathogen is retained in the polymeric aerogel and air is allowed to pass through the polymeric aerogel. Aspect 31 is the air-permeable filter material of aspect 30, wherein the airborne pathogen is a virus, a bacteria, a fungi, or a protozoa. Aspect 32 is the air-permeable filter material of aspect 31, wherein the airborne pathogen is a virus, and wherein the virus is preferably an adenovirus, alphavirus, calicivirus, coronavirus, distemper virus, Ebola virus, enterovirus, flavivirus, hepatitis virus, herpesvirus, infectious peritonitis virus, leukemia virus, Marburg virus, Norwalk virus, orthomyxovirus, papilloma virus, parainfluenza virus., the, paramyxovirus, parvovirus, pestivirus, picorna virus, pox virus, rabies virus, reovirus polypeptides, retrovirus, rotavirus, and vaccinia virus. Aspect 33 is the air-permeable filter material of aspect 32, wherein the airborne pathogen is a coronavirus. Aspect 34 is the air-permeable filter material of aspect 33, wherein the coronavirus is MERS-CoV, SARS-CoV, or SARS-CoV-2, preferably SARS-CoV-2. Aspect 35 is the air-permeable filter material of aspect 31, wherein the airborne pathogen is a bacteria, and wherein the bacterial is preferably Actinomyces, Bacillus, Bacteroides, Bordetella, Bartonella, Borrelia, Brucella, Campylobacter, Capnocytophaga, Chlamydia, Corynebacterium, Coxiella, Dermatophilus, Enterococcus, Ehrlichia, Escherichia, Francisella, Fusobacterium, Haemobartonella, Haemophilus, Helicobacter, Klebsiella, L-form bacteria, Leptospira, Listeria, Mycobacteria, Mycoplasma, Neisseria, Neorickettsia, Nocardia, Pasteurella, Peptococcus, Peptostreptococcus, Pneumococcus, Proteus, Pseudomonas, Rickettsia, Rochalimaea polypeptides, Salmonella, Shigella, Staphylococcus, group A streptococcus, group B streptococcus, Treponema, and Yersinia. Aspect 36 is the air-permeable filter material of aspect 31, wherein the airborne pathogen is a fungi, and wherein the fungi is preferably Absidia, Acremonium, Alternaria, Aspergillus, Basidiobolus, Bipolaris, Blastomyces, Candida, Coccidioides, Conidiobolus, Cryptococcus, Curvalaria, Epidermophyton, Exophiala, Geotrichum, Histoplasma, Madurella, Malassezia, Microsporum, Moniliella, Mortierella, Mucor, Paecilomyces, Penicillium, Phialemonium, Phialophora, Prototheca, Pseudallescheria, Pseudomicrodochium, Pythium, Rhinosporidium, Rhizopus, Scolecobasidium, Sporothrix, Stemphylium, Trichophyton, Trichosporon, and Xylohypha. Aspect 37 is the air-permeable filter material of aspect 31, wherein the airborne pathogen is a protozoa, and wherein the protozoa is preferably Babesia, Balantidium, Besnoitia, Cryptosporidium, Eimeria, Encephalitozoon, Entamoeba, Giardia, Hammondia, Hepatozoon, Isospora, Leishmania, Microsporidia, Neospora, Nosema, Pentatrichomonas, Plasmodium. Aspect 38 is the air permeable filter material of any one of aspects 1 to 37, wherein the polymeric aerogel comprises micropores, mesopores, or macropores, or a combination thereof. Aspect 39 is the air permeable filter material of aspect 38, wherein at least 10%, 50%, 75%, 95%, or 100% of the aerogel's pore volume is made up of micropores. Aspect 40 is the air permeable filter material of any one of aspects 38 to 39, wherein at least 10%, 50%, 75%, 95%, or 100% of the aerogel's pore volume is made up of mesopores. Aspect 41 is the air permeable filter material of any one of aspects 38 to 40, wherein at least 10%, 50%, 75%, 95%, or 100% of the aerogel's pore volume is made up of macropores. Aspect 42 is the air permeable filter material of aspect 41, wherein less than 90%, 80%, 70%, 60%, 50%, 40%, 30% 20%, 10% or less than 5% of the aerogel's pore volume is made up of micropores and/or mesopores. Aspect 43 is the air permeable filter material of any one of claims 38 to 42, wherein the polymeric matrix has an average pore size of 2 nanometers (nm) to 50 nm in diameter. Aspect 44 is the air permeable filter material of any one of aspects 38 to 42, wherein the polymeric matrix has an average pore size of greater than 50 nanometers (nm) to 5000 nm in diameter, preferably 1,000 nm to 1,400 nm, or more preferably around 1,200 nm. Aspect 45 is the air permeable filter material of aspect 44, wherein the polymeric matrix has an average pore size of 100 nm to 800 nm, preferably 100 nm to 500 nm, more preferably from 150 nm to 400 nm, even more preferably from 200 nm to 300 nm, still more preferably from 225 nm to 275 nm. Aspect 46 is the air permeable filter material of any one of aspects 1 to 45, wherein the polymeric aerogel is an organic polymeric aerogel. Aspect 47 is the air permeable filter material of any one of aspects 1 to 46, wherein the polymeric aerogel is a polyimide aerogel. Aspect 48 is the air permeable filter material of any one of aspects 1 to 46, wherein the polymeric aerogel is a polyamide aerogel, a polyaramid aerogel, a polyurethane aerogel, a polyuria aerogel, or a polyester aerogel. Aspect 49 is the air permeable filter material of any one of aspects 1 to 48, further comprising a support film or layer at least partially penetrating the polymer matrix of the aerogel. Aspect 50 is the air permeable filter material of aspect 49, wherein the support film is a fiber support. Aspect 51 is the air permeable filter material of aspect 50, wherein the fiber support is a woven fiber support, knitted fiber support, non-woven fiber support or a paper. Aspect 52 is the air permeable filter material of any one of aspects 1 to 51, wherein the material is in the form of a film or layer having a thickness of 0.01 millimeters (mm) to 1000 mm thick, or from 0.025 mm to 100 mm thick, or from 0.050 mm to 10 mm thick, or from 0.100 mm to 2 mm thick, or from 0.125 mm to 1.5 mm. Aspect 53 is the air permeable filter material of any one of aspects 1 to 52, wherein the material is re-useable, washable, and/or capable of being disinfected and reused.

Aspect 54 is a method of removing airborne pathogens, the method comprising passing air comprising the airborne pathogen through at least a portion of the air permeable filter material of any one of aspects 1 to 53 such that the airborne pathogen is retained in the polymeric aerogel and air is allowed to pass through the polymeric aerogel. Aspect 55 is the method of aspect 54, wherein the airborne pathogen is a virus, a bacteria, a fungi, or a protozoa. Aspect 56 is the method of aspect 55, wherein the airborne pathogen is a virus, and wherein the virus is preferably an adenovirus, alphavirus, calicivirus, coronavirus, distemper virus, Ebola virus, enterovirus, flavivirus, hepatitis virus, herpesvirus, infectious peritonitis virus, leukemia virus, Marburg virus, Norwalk virus, orthomyxovirus, papilloma virus, parainfluenza virus., the, paramyxovirus, parvovirus, pestivirus, picorna virus, pox virus, rabies virus, reovirus polypeptides, retrovirus, rotavirus, and vaccinia virus. Aspect 57 is the method of aspect 56, wherein the airborne pathogen is a coronavirus. Aspect 58 is the method of aspect 57, wherein the coronavirus is MERS-CoV, SARS-CoV, or SARS-CoV-2, preferably SARS-CoV-2. Aspect 59 is the method of aspect 55, wherein the airborne pathogen is a bacteria, and wherein the bacterial is preferably Actinomyces, Bacillus, Bacteroides, Bordetella, Bartonella, Borrelia, Brucella, Campylobacter, Capnocytophaga, Chlamydia, Corynebacterium, Coxiella, Dermatophilus, Enterococcus, Ehrlichia, Escherichia, Francisella, Fusobacterium, Haemobartonella, Haemophilus, Helicobacter, Klebsiella, L-form bacteria, Leptospira, Listeria, Mycobacteria, Mycoplasma, Neisseria, Neorickettsia, Nocardia, Pasteurella, Peptococcus, Peptostreptococcus, Pneumococcus, Proteus, Pseudomonas, Rickettsia, Rochalimaea polypeptides, Salmonella, Shigella, Staphylococcus, group A streptococcus, group B streptococcus, Treponema, and Yersinia. Aspect 60 is the method of aspect 55, wherein the airborne pathogen is a fungi, and wherein the fungi is preferably Absidia, Acremonium, Alternaria, Aspergillus, Basidiobolus, Bipolaris, Blastomyces, Candida, Coccidioides, Conidiobolus, Cryptococcus, Curvalaria, Epidermophyton, Exophiala, Geotrichum, Histoplasma, Madurella, Malassezia, Microsporum, Moniliella, Mortierella, Mucor, Paecilomyces, Penicillium, Phialemonium, Phialophora, Prototheca, Pseudallescheria, Pseudomicrodochium, Pythium, Rhinosporidium, Rhizopus, Scolecobasidium, Sporothrix, Stemphylium, Trichophyton, Trichosporon, and Xylohypha. Aspect 61 is the method of aspect 55, wherein the airborne pathogen is a protozoa, and wherein the protozoa is preferably Babesia, Balantidium, Besnoitia, Cryptosporidium, Eimeria, Encephalitozoon, Entamoeba, Giardia, Hammondia, Hepatozoon, Isospora, Leishmania, Microsporidia, Neospora, Nosema, Pentatrichomonas, Plasmodium.

Aspect 62 is a method of disinfecting the air permeable filter material of any one of claims 1 to 53, the method comprising subjecting the material to a disinfectant solution or spray capable of killing an airborne pathogen. Aspect 63 is the method of aspect 62, wherein the airborne pathogen is a virus, a bacteria, a fungi, or a protozoa. Aspect 64 is a method of making the air permeable filter material of any one of aspects 1 to 53, the method comprising: (a) providing a monomer or a combination of monomers to a solvent to form a solution; (b) polymerizing the monomers in the solution to form a polymer gel matrix; and (c) subjecting the polymer gel matrix to conditions sufficient to remove liquid from the polymer gel matrix to form an aerogel having a polymeric matrix comprising an open-cell structure. Aspect 65 is the method of aspect 64, wherein step (b) further comprises adding a curing agent to the solution to reduce the solubility of polymers formed in the solution and to form macropores in the gel matrix, the formed macropores containing liquid from the solution. Aspect 66 is the method of any one of aspects 64 to 65, the method further comprising: covering or immersing a support film in the solution in step (a) or (b) such that the support film is attached to the resulting polymer gel matrix and ultimately to the produced aerogel matrix. Aspect 67 is the method of any one of aspects 64 to 66, further comprising casting the polymer gel matrix in step (b) onto a support such that a layer of the polymeric gel matrix is comprised on the support, wherein the aerogel in step (c) is in the form of a film. Aspect 68 is the method of aspect 67, further comprising crushing, grinding, or milling the film to produce aerogel particles.

In some aspects, the air-permeable filter material of the present invention can have a particular bacterial filtration efficiency. In some aspects, the bacterial filtration efficiency can be greater than or equal to 95%, greater than or equal to 98%, greater than or equal to 99%, greater than or equal to 99.5%, greater than or equal to 99.9%, greater than or equal to 99.99%, greater than or equal to 99.999%, greater than or equal to 99.9999%, or greater than or equal to 99.99999%. In some aspects, the bacterial filtration efficiency of the air-permeable filter material can be less than or equal to 99.99995%, less than or equal to 99.9999%, less than or equal to 99.999%, less than or equal to 99.99%, less than or equal to 99.9%, less than or equal to 99.5%, less than or equal to 99%, or less than or equal to 98%. In some aspects, the air-permeable filter material of the present invention can have a bacterial filtration efficiency of greater than or equal to 95% and less than or equal to 99.99995%. Bacterial filtration efficiency can be measured according to ASTM F2101 as the percent of bacteria (e.g., Staphylococcus aureus) collected downstream of an air-permeable filter material of the present invention versus the bacteria provided upstream of the air-permeable filter material of the present invention in an aerosol initially comprising 1 million bacterial units at a face velocity of 12.5 cm/s and a flow rate of 30 liters per minute over an area of 40 cm².

In other aspects, the air-permeable filter material of the present invention can have a particular viral filtration efficiency. In some aspects, the viral filtration efficiency can be greater than or equal to 95%, greater than or equal to 98%, greater than or equal to 99%, greater than or equal to 99.5%, greater than or equal to 99.9%, greater than or equal to 99.99%, greater than or equal to 99.999%, greater than or equal to 99.9999%, or greater than or equal to 99.99999%. In some aspects, the viral filtration efficiency of the filter media can be less than or equal to 99.99995%, less than or equal to 99.9999%, less than or equal to 99.999%, less than or equal to 99.99%, less than or equal to 99.9%, less than or equal to 99.5%, less than or equal to 99%, or less than or equal to 98%. In some aspects, the air-permeable filter material of the present invention can have a viral filtration efficiency of greater than or equal to 95% and less than or equal to 99.99995%. The viral filtration efficiency can be measured according to ASTM F2101 as the percent of viruses (e.g., Phi X174 bacteriophase) collected downstream of the air-permeable filter material of the present invention versus the viruses provided upstream of the air-permeable filter material of the present invention in an aerosol initially comprising 107 plaque-forming units of the virus at a flow rate of 30 liters per minute and face velocity of 12.5 cm/s over an area of 40 cm².

In some aspects, the air-permeable filter material of the present invention can have a particular sub-micron particulate filtration efficiency. In some aspects, the sub-micron particulate filtration efficiency can be greater than or equal to 95%, greater than or equal to 98%, greater than or equal to 99%, greater than or equal to 99.5%, greater than or equal to 99.9%, greater than or equal to 99.99%, greater than or equal to 99.999%, greater than or equal to 99.9999%, or greater than or equal to 99.9999%. In some aspects, the sub-micron particulate filtration efficiency of the air-permeable filter material can be less than or equal to 99.99995%, less than or equal to 99.9999%, less than or equal to 99.999%, less than or equal to 99.99%, less than or equal to 99.9%, less than or equal to 99.5%, less than or equal to 99%, or less than or equal to 98%. In some aspects, the air-permeable filter material of the present invention can have a sub-micron particulate filtration efficiency of greater than or equal to 95% and less than or equal to 99.99995%. Sub-micron particulate filtration efficiency can be measured according to ASTM F1215 (e.g., using 0.1 micron Latex spheres).

In some aspects, the air-permeable filter material of the present invention can be tested to determine inhalation and exhalation resistance. In some aspects, the resistance to inhalation at 85 L/min (mm water column) can be 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or less than 35 mm H₂O or any range therein. In other aspects, the resistance to inhalation at 85 L/min (mm water column) can be 35 mm H₂O or greater (e.g., 36, 37, 38, 39, 40, 41, 42, 43, 44, 45, 46, 47, 48, 49, 50, 51, 52, 53, 54, 55, 56, 57, 58, 59, 60, or more, or any range therein). In some aspects, the resistance to exhalation at 85 L/min (mm water column) can be 5, 6, 7, 8, 9, 10, 11, 12, 13, 14, 15, 16, 17, 18, 19, 20, 21, 22, 23, 24, or less than 25 mm H₂O or any range therein. In other aspects, the resistance to exhalation at 85 L/min (mm water column) can be 25 mm H₂O or greater (e.g., 26, 27, 28, 29, 30, 31, 32, 33, 34, 35, 36, 37, 38, 39, 40, 41, 42, 43, 44, 45, 46, 47, 48, 49, 50, or more, or any range therein). The inhalation and exhalation resistance test can be measured according to ASTM F2100-Standard Specification For Performance of Materials Used In Medical face Masks. A value of 35 mm H₂O (pressure) or greater can indicate that resistance to inhalation is not preferred. A value of 25 mm H₂O (pressure) or greater can indicate that resistance to exhalation is not preferred.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions of the invention can be used to achieve methods of the invention.

The following includes definitions of various terms and phrases used throughout this specification.

The term “aerogel” refers to a class of materials that are generally produced by forming a gel, removing a mobile interstitial solvent phase from the pores, and then replacing it with a gas or gas-like material. By controlling the gel and evaporation system, density, shrinkage, and pore collapse can be minimized. Aerogels of the present invention can include macropores, mesopores, and/or micropores. In preferred aspects, the majority (e.g., more than 50%) of the aerogel's pore volume can be made up of macropores. In other alternative aspects, the majority of the aerogel's pore volume can be made up of mesopores and/or micropores such that less than 50% of the aerogel's pore volume can be made up of macropores. In some embodiments, the aerogels of the present invention can have low bulk densities (about 0.75 g/cm³ or less, preferably about 0.01 to 0.5 g/cm³), high surface areas (generally from about 10 to 1,000 m²/g and higher, preferably about 50 to 1000 m²/g), high porosity (about 20% and greater, preferably greater than about 85%), and/or relatively large pore volume (more than about 0.3 mL/g, preferably about 1.2 mL/g and higher).

The presence of macropores, mesopores, and/or micropores in the aerogels of the present invention can be determined by mercury intrusion porosimetry (MIP) and/or gas physisorption experiments. The MIP test can be used to measure mesopores and macropores (i.e., American Standard Testing Method (ASTM) D4404-10, Standard Test Method for Determination of Pore Volume and Pore Volume Distribution of Soil and Rock by Mercury Intrusion Porosimetry). Gas physisorption experiments can be used to measure micropores (i.e., ASTM D1993-03(2008) Standard Test Method for Precipitated Silica—Surface Area by Multipoint BET Nitrogen).

The term “non-woven” is defined as material made of fibers that does not have a woven or interlaced architecture using continuous fibers. However, the non-woven fibrous region of the supports of the present invention may have some inadvertent cross-over of some of the individual filaments, such cross-over does not change the non-woven structure of the fibrous region and is not a designed continuous aspect of the material.

The terms “impurity” or “impurities” refers to unwanted substances in a feed fluid that are different than a desired filtrate and/or are undesirable in a filtrate. In some instances, impurities can be solid, liquid, gas, or supercritical fluid. In some embodiments, an aerogel can remove some or all of an impurity.

The term “desired substance” or “desired substances” refers to wanted substances in a feed fluid that are different than the desired filtrate. In some instances, the desired substance can be solid, liquid, gas, or supercritical fluid. In some embodiments, an aerogel can remove some or all of a desired substance.

The use of the word “a” or “an” when used in conjunction with the terms “comprising,” “including,” “containing,” or “having” in the claims and/or the specification may mean “one,” but it is also consistent with the meaning of “one or more,” “at least one,” and “one or more than one.”

The terms “about” or “approximately” are defined as being close to as understood by one of ordinary skill in the art. In one non-limiting embodiment, the terms are defined to be within 10%, preferably within 5%, more preferably within 1%, and most preferably within 0.5%.

The use of the term “or” in the claims is used to mean “and/or” unless explicitly indicated to refer to alternatives only or the alternatives are mutually exclusive, although the disclosure supports a definition that refers to only alternatives and “and/or.”

The terms “wt. %,” “vol. %,” or “mol. %” refers to a weight, volume, or molar percentage of a component, respectively, based on the total weight, the total volume, or the total moles of material that includes the component. In a non-limiting example, 10 moles of component in 100 moles of the material is 10 mol. % of component.

The term “substantially” and its variations are defined to include ranges within 10%, within 5%, within 1%, or within 0.5%.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The aerogels and processes of making and using the aerogels of the present invention can “comprise,” “consist essentially of,” or “consist of” particular ingredients, components, compositions, steps, etc., disclosed throughout the specification. With respect to the transitional phase “consisting essentially of,” in one non-limiting aspect, a basic and novel characteristic of the air permeable filter materials of the present invention is that they can be used in PPE (e.g., facial or surgical masks) to filter our undesired airborne pathogens while allowing an acceptable amount of air to pass through to make the material a breathable filter that efficiently filters out the undesired pathogens.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1A is an illustration of an air-permeable filter material of the present invention in the form of a polymeric aerogel having a polymeric matrix comprising an open-cell structure. The aerogel is in the form of a film.

FIG. 1B is an illustration of an air-permeable filter material of the present invention in the form of a polymeric aerogel having a polymeric matrix comprising an open-cell structure that is adhesively attached to a support. The aerogel is in the form of a film.

FIG. 1C is an illustration of an air-permeable filter material of the present invention in the form of a polymeric aerogel having a polymeric matrix comprising an open-cell structure. The polymeric aerogel is in particulate form.

FIG. 1D is an illustration of an air-permeable filter material of the present invention in the form of a polymeric aerogel having a polymeric matrix comprising an open-cell structure. The polymeric aerogel is in particulate form and is contained within a support material.

FIG. 2A is an illustration of a PPE (face mask) that includes an air-permeable filter material of the present invention.

FIG. 2B is an illustration of a PPE (face mask) that includes an air-permeable filter material of the present invention positioned on a user's face.

FIG. 3 is an illustration of a PPE (face mask) that includes an air-permeable filter material of the present invention.

FIG. 4 is an illustration of a PPE (face mask) that includes an air-permeable filter material of the present invention.

FIG. 5 is an illustration of a cartridge that can be used in PPE (e.g., face mask). The cartridge includes an air-permeable filter material of the present invention.

FIG. 6 is an illustration of a PPE (face mask) that can include a cartridge having an air-permeable filter material of the present invention.

FIG. 7 is an illustration of various types of face masks that can include an air-permeable filter material of the present invention.

FIG. 8 is an illustration of a heat and/or air conditioning air filter that can include an air-permeable filter material of the present invention.

FIG. 9 is an illustration of an embodiment of an internally reinforced aerogel having the support positioned at about the midline of the aerogel.

FIG. 10 is an illustration of embodiment of an internally reinforced aerogel having the support positioned at internal offset position in the aerogel.

FIG. 11 is an illustration of an embodiment of an internally reinforced aerogel having the support positioned at the edge of the aerogel.

FIG. 12 is an illustration of an embodiment of an internally reinforced aerogel having the support positioned partially penetrating the aerogel.

FIG. 13 is an illustration of an embodiment of an aerogel laminate comprising a plurality or reinforced aerogels formed into a multiplayer laminate structure.

FIG. 14 is a distribution of pore size diameter for a first non-limiting aerogel of the present invention.

FIG. 15 is a distribution of pore size diameter for a second non-limiting aerogel of the present invention.

FIG. 16 is a distribution of pore size diameter for a third non-limiting aerogel of the present invention.

FIG. 17 is an image of a produced air-permeable filter material of the present invention having polymeric aerogel film with a polymeric matrix comprising an open-cell structure that is adhesively attached to a fiber scrim support.

FIG. 18 is an image of various parts of an air-filter mask of the present invention that includes a fiber scrim supported aerogel film, a strip of foam that can be used to provide a cushion to a user's nose, and fabric that can be used to support the fiber scrim supported aerogel film and strip of foam.

FIG. 19 is an image of an assembled air-filter mask of the present invention.

FIG. 20 is an image of another assembled air-filter mask of the present invention.

While the invention is susceptible to various modifications and alternative forms, specific embodiments thereof are shown by way of example in the drawings and may herein be described in detail. The drawings may not be to scale.

### DETAILED DESCRIPTION

A discovery has been made that provides a solution to at least some of the problems associated with PPE and/or other equipment that uses filters to filter out undesired particles from air. In one aspect, the solution resides in the use of polymeric aerogels that have a polymeric matrix with an open-cell structure in filtration applications such as PPE equipment (e.g., face or surgical masks). The polymeric aerogels used to make the filter material have a sufficient amount of structural integrity that allows the material to be washed or disinfected or sterilized and reused. This provides an advantage in that it can reduce or prevent PPE shortages such as the shortages seen in today's society, which is dealing with the novel coronavirus (SARS-CoV-2) pandemic. Further, the porous structure of the polymeric aerogels allows for a sufficient amount of air to flow through the aerogel while preventing or limited the flow of viral or bacterial particles, such as, for example particles of SARS-CoV-2.

These and other non-limiting aspects of the present invention are discussed in further detail in the following sections.

### A. Air-Permeable Filter Material and Air-Filtration Devices

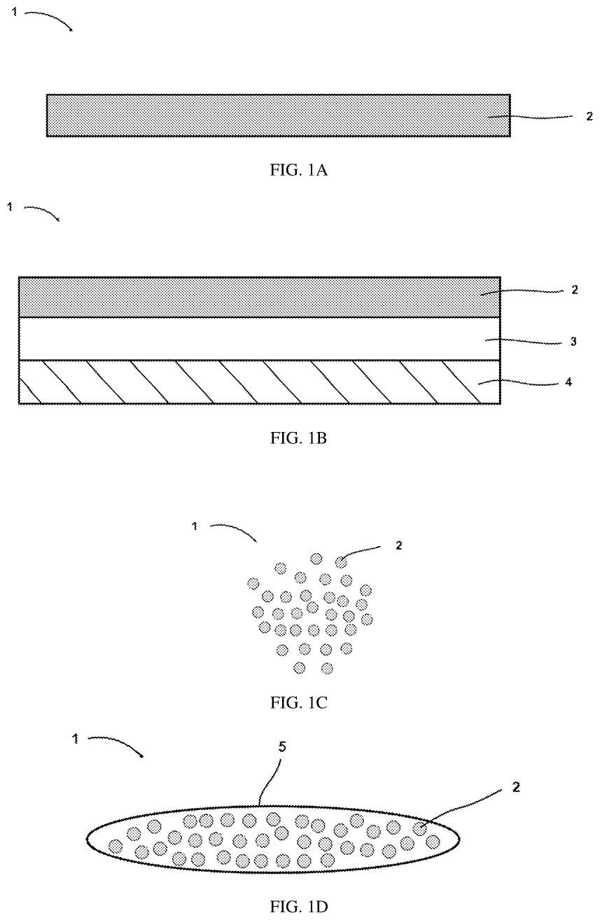

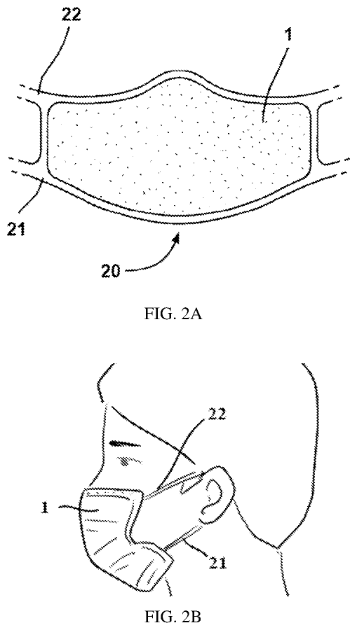

Referring to FIGS. 1A-1D, air-permeable filter materials of the present invention are disclosed. FIG. 1A illustrates an air-permeable filter material 1 having a polymeric aerogel 2 comprising a polymeric matrix that includes an open-cell structure. The aerogel 2 is in the form of a film. The thickness of the film can be designed to be useable in PPE such as face masks. In some aspects, the thickness of the film can be 0.01 millimeters (mm) to 1000 mm thick, or 0.025 mm to 100 mm thick, or 0.050 mm to 10 mm thick, or 0.100 mm to 2 mm thick, or from 0.125 mm to 1.5 mm. In preferred instances, the thickness can be 0.100 mm to 2 mm thick, or more preferably 0.125 mm to 1.5 mm thick. Processes for making the aerogel film are provided below. The shape of the air filter material 1 can be any shape desired and/or based on the structure of the equipment the filter material is being used with (e.g., circular, triangular, rectangular, square, irregular shape, random shape, etc.).

FIG. 1B illustrates an air-permeable filter material 1 having a polymeric aerogel 2 comprising a polymeric matrix that includes an open-cell structure. The aerogel 2 is in the form of a film and attached to a support or substrate 4 (support and substrate can be used interchangeably throughout this specification) through an adhesive 3. The support 4 can include a paper material, a fibrous material, a metal material, a thermoplastic material, or a thermoset material. In some aspects, the support 4 can be a fiber support, preferably, a woven fiber substrate, a knitted fiber substrate, non-woven fiber substrate, or a paper substrate. The support 4 can be in the form of a scrim. Non-limiting examples of adhesives that can be used include epoxy resins, ethylene-vinyl acetate. phenol formaldehyde resins, polyamide resins, polyester resins, polyethylene resins, polypropylene resins, polysulfide resins, polyurethane resins, polyvinyl acetate (PVA), polyvinyl alcohol, polyvinyl chloride (PVC), polyvinyl chloride emulsion (PVCE), polyvinylpyrrolidone, rubber cement, acrylate and/or acrylic acid-based adhesives, silicones-based adhesives, or silyl modified polymers. In particular instances, a polyester-based resin can be used. The supported filter material 1 in FIG. 1B can be prepared by applying adhesive 3 to at least a portion of the aerogel 2 and/or a portion of the substrate 4 and pressing the substrate 4 and aerogel 2 together for a sufficient time to adhere the aerogel 2 to the substrate 4. In particular instances, the substrate 4 can be dipped or coated in the adhesive 3 and then the substrate 4 and aerogel 2 can be pressed together. In instances where a scrim is used as a substrate 4, the scrim can be coated or dipped into the adhesive 3 and then then scrim and aerogel 2 can be pressed together. Although not shown, multiple layers of the aerogel 2 and/or substrate 4 can be used to create a multi-layered or laminate based filter material 1. The shape of the air filter material 1 and substrate 4 can be any shape desired and/or based on the structure of the equipment the filter material is being used with (e.g., circular, triangular, rectangular, square, irregular shape, random shape, etc.).

FIG. 1C illustrates an air permeable filter 1 in which the aerogel 2 is in particulate form. The particles of the aerogel 2 can be used as the filter material by packing the particles 2 together. FIG. 1D illustrates particles of the aerogel 2 packed together in a substrate material 5. The substrate material 5 can be a permeable material that can allow air through such that the air to be filtered comes into contact with the aerogel particles 2. In some aspects, the substrate material 5 can be formed such that the aerogel particles 2 are retained within a space or pocket formed by the material 5. In other instances, the particles 2 can be dispersed throughout the substrate material 5. The substrate material 5 can include a paper material, a fibrous material, a metal material, a thermoplastic material, or a thermoset material. In some aspects, the substrate material 5 can be a fiber support, preferably, a woven fiber substrate, a knitted fiber substrate, non-woven fiber substrate, or a paper substrate. Further, other particulate material (not shown) can be mixed with the aerogel particles 2. In still other aspects, the substrate material 5 can be the aerogel film or films of the present invention, and the film or films of the present invention can be positioned such that they form the space or pocket for the aerogel particles 2. This combination of aerogel films and particles can provide an enhanced or dual filtration effect. The other particulate material can be filler material, desiccant material, filter material, anti-viral material, anti-bacterial material, anti-fungal material, anti-protozoa material, etc. In some aspects, the other particulate material can be filter material such as activated carbon or charcoal containing filter material. Processes for making the aerogel particulate material are provided below. The shape of the particles 2 can be any shape desired and/or based on the structure of the equipment the filter material is being used with (e.g., spherical, triangular, rectangular, square, random shape, irregular shape, etc.). In preferred aspects, the particles have a substantially or overall spherical shape. Further, the substrate material 5 can be any shape desired and/or based on the structure of the equipment the filter material is being used with (e.g., circular, triangular, rectangular, square, etc.).

Referring to FIG. 2A, a face mask 20 that can be used as PPE is disclosed. The face mask 20 can include a first strap 21 and a second strap 22, which can be used to secure the mask 20 on a person's face by placing at least a portions of the first and second straps 21 and 22 around the person's head or ear. FIG. 2B provides an illustrate of a face mask 20 on a person's face. The straps 21 and 22 can include flexible material, non-flexible material, or a combination thereof. The straps 21 and 22 can attach to portions of an air-permeable filter material 1 of the present invention.

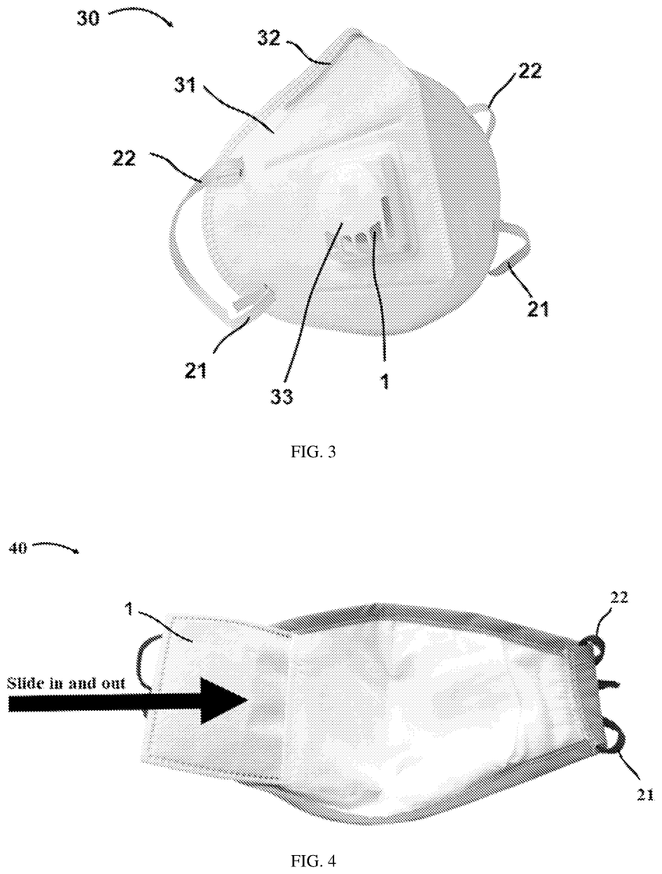

Referring to FIG. 3, a face mask 30 is disclosed in which a filter material 1 of the present invention can be placed in a particular position with a valve 33. In other instances, the valve 33 may not include a filter material 1 of the present invention. Instead, at least a portion of the mask 30, the portion represented by 31, can be made of the filter material 1 of the present invention. The mask 30 can include a malleable piece of material 32 (e.g., metal) that can be used to secure the mask 30 to the bridge of a person's nose.

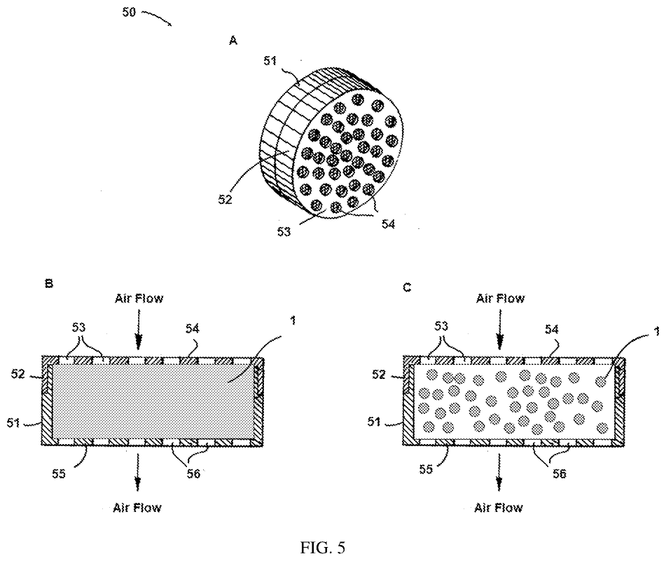

Referring to FIG. 4, a face mask 40 is disclosed in which the filter material 1 of the present invention can be removed by sliding the material 1 into and out of a pocket formed in the mask 40. This set up can be useful in instances where replacement of the filter material 1 is desired but replacement of the mask is not desired. This can result in a mask that has a replaceable filter material 1, which can be replaced as needed.

Referring to FIG. 5, a cartridge 50 is provided, which can be used with an air filter mask of the present invention. The cartridge 50 can include a lower portion 51 and an upper portion 52 that can be connected together to form an internal space (not shown). The connection can be a press-fit connection, a threaded connection, an adhesive connection, or any other type of connection known in the art. In instances, where the cartridge 50 is to be reused, then a press-fit or threaded connection can be preferred, and the portions 51 and 52 can be separated and reloaded with air filter material 1 of the present invention. In instances where the cartridge 50 may not be reused, then an adhesive connection may be preferred, and the cartridge 50 can be discarded after use. It is also contemplated that the cartridge 50 may only include a single portion (not shown) or additional portions (not shown). In certain aspects, the cartridge can include a perforated upper surface 53 and a perforated lower surface 56. The perforated surfaces 53 and 56 can include apertures 54 on the upper surface 53 and apertures 55 on the lower surface 56. The apertures 54 and 55 can allow for air to flow through the cartridge and to come into contact with the air filter material 1 of the present invention. The air filter material 1 can be any air filter material of the present invention. By way of example,

FIG. 5B illustrates an air filter material 1 that can be in the form of a film, whereas FIG. 5C represents an air filter material 1 that can be in particulate form. The cartridge 50 is represented as having a circular shape. Other shapes are contemplated (e.g., triangular, rectangular, square, irregular shape, random shape, etc.) and can be designed as desired.

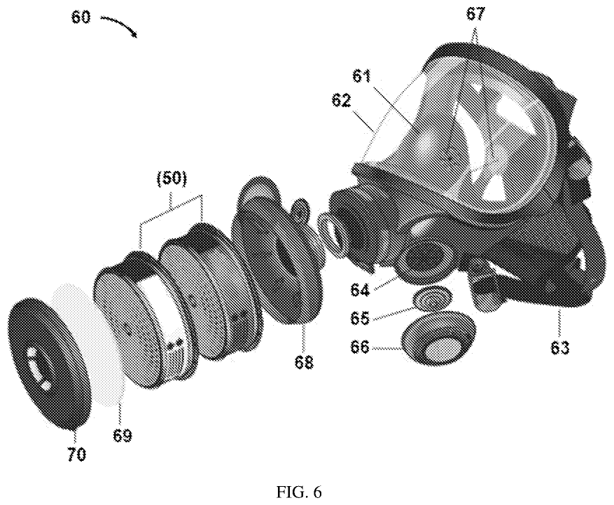

FIG. 6 represents various parts of a face mask 60 that can utilize a cartridge 50 or multiple cartridges 50 of the present invention. In addition to the cartridges 50, the face mask 60 can include a pre-filter material 69. In certain aspects, the pre-filter 69 can also be an air filter material 1 of the present invention. In other instances, the pre-filter material 69 can be made from a material other than a polymer aerogel of the present invention (e.g., fibrous material). The face mask 60 can also include any one, any combination of, or all of a mask body 61, a visor (e.g., transparent material such as polycarbonate or glass) 62, elastic material 63 to secure to a user's head, an exhalation valve seat 64, an exhalation membrane 65, a protective cap 66, an inhalation valve 67, an adapter 68, and a pre-filter holder 70.

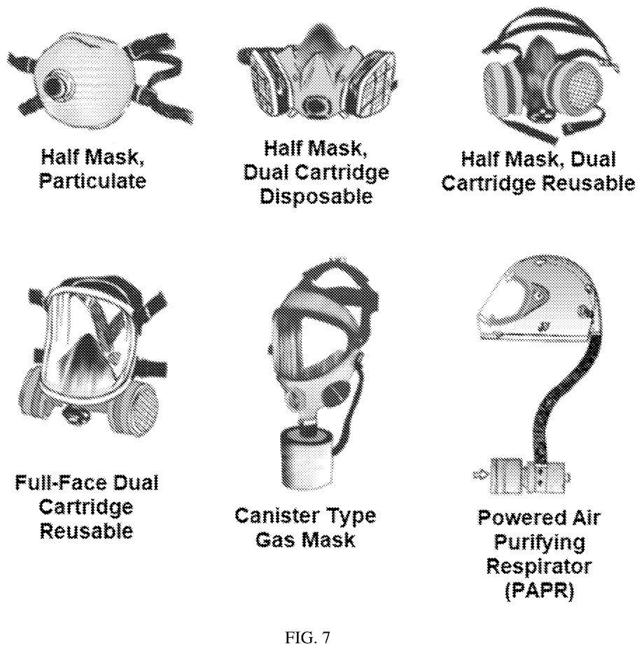

FIG. 7 illustrates non-limiting examples of air-filtration masks that can include the air-filter material 1 of the present invention.

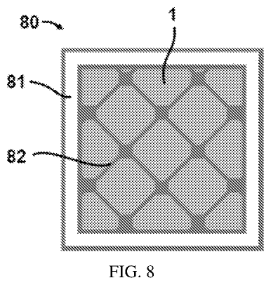

FIG. 8 is an illustration of a heat and/or air conditioning air filter 80 that can include an air-permeable filter material 1 of the present invention. The air filter 80 can include a border 81 and a support structure 82. The border 81 and support structure 82 can each be made of any desired material (e.g., plastic, metal, fibrous material (e.g., paper, cardboard, etc.), etc.). In some preferred aspects, the border can be made of paper or cardboard. In some preferred aspects, the support structure can be made of metal such as malleable wires or wire mesh. The shape of the air filter 80 in FIG. 8 is square. However, other shapes are contemplated such as rectangular, circular, triangular, irregular shape, random shape, etc. In preferred aspects, square and rectangular shapes can be used. The air filter 80 can be used in homes and office buildings such as in wall or ceiling or floor vent spaces. The air filter 80 can also be used in automotive air circulation systems (e.g., automobile air conditioning and/or heater units or automotive filter systems).

### B. Types of Polymeric Aerogels

The polymeric aerogel comprising the polymeric matrix used in the air-permeable filter material 1 of the present invention can include organic materials, inorganic materials, or a mixture thereof, and have matrices that include macropores, mesopores, or micropores, a combination thereof. The aerogels or wet gels used to prepare the aerogels may be prepared by any known gel-forming techniques, for example adjusting the pH and/or temperature of a dilute metal oxide sol to a point where gelation occurs. Aerogels can be made from polyacrylates, polystyrenes, polyacrylonitriles, polyurethanes, polysiloxanes, polyimides, polyamides, polyaramids, polyfurfural alcohol, phenol furfuryl alcohol, melamine formaldehydes, resorcinol formaldehydes, cresol formaldehyde, phenol formaldehyde, polyvinyl alcohol dialdehyde, polycyanurates, polyacrylamides, various epoxies, agar, agarose, and the like. In particular embodiments the aerogel is a polyimide aerogel.

Polyimides are a type of polymer with many desirable properties. Polyimide polymers include a nitrogen atom in the polymer backbone, where the nitrogen atom is connected to two carbonyl carbons, such that the nitrogen atom is somewhat stabilized by the adjacent carbonyl groups. A carbonyl group includes a carbon, referred to as a carbonyl carbon, which is double bonded to an oxygen atom. Polyimides are usually considered an AA-BB type polymer because usually two different classes of monomers are used to produce the polyimide polymer. Polyimides can also be prepared from AB type monomers. For example, an aminodicarboxylic acid monomer can be polymerized to form an AB type polyimide. Monoamines and/or mono anhydrides can be used as end capping agents if desired.

One class of polyimide monomer is usually a diamine, or a diamine monomer. The diamine monomer can also be a diisocyanate, and it is to be understood that an isocyanate could be substituted for an amine in this description, as appropriate. There are other types of monomers that can be used in place of the diamine monomer, as known to those skilled in the art. The other type of monomer is called an acid monomer, and is usually in the form of a dianhydride. In this description, the term “di-acid monomer” is defined to include a dianhydride, a tetraester, a diester acid, a tetracarboxylic acid, or a trimethylsilyl ester, all of which can react with a diamine to produce a polyimide polymer. Dianhydrides are to be understood as tetraesters, diester acids, tetracarboxylic acids, or trimethylsilyl esters that can be substituted, as appropriate. There are also other types of monomers that can be used in place of the di-acid monomer, as known to those skilled in the art.

Because one di-acid monomer has two anhydride groups, different diamino monomers can react with each anhydride group so the di-acid monomer may become located between two different diamino monomers. The diamine monomer contains two amine functional groups; therefore, after the first amine functional group attaches to one di-acid monomer, the second amine functional group is still available to attach to another di-acid monomer, which then attaches to another diamine monomer, and so on. In this manner, the polymer backbone is formed. The resulting polycondensation reaction forms a polyamic acid.

The polyimide polymer is usually formed from two different types of monomers, and it is possible to mix different varieties of each type of monomer. Therefore, one, two, or more di-acid monomers can be included in the reaction vessel, as well as one, two or more diamino monomers. The total molar quantity of di-acid monomers is kept about the same as the total molar quantity of diamino monomers if a long polymer chain is desired. Because more than one type of diamine or di-acid can be used, the various monomer constituents of each polymer chain can be varied to produce polyimides with different properties. For example, a single diamine monomer AA can be reacted with two di-acid co monomers, B₁B₁ and B₂B₂, to form a polymer chain of the general form of (AA-B₁B₁)ₓ-(AA-B₂B₂)_{y} in which x and y are determined by the relative incorporations of B₁B₁ and B₂B₂ into the polymer backbone. Alternatively, diamine co-monomers A₁A₁ and A₂A₂ can be reacted with a single di-acid monomer BB to form a polymer chain of the general form of (A₁A₁-BB)ₓ-(A₂A₂-BB)_{y}. Additionally, two diamine co-monomers A₁A₁ and A₂A₂ can be reacted with two di-acid co-monomers B₁B₁ and B₂B₂ to form a polymer chain of the general form (A₁A₁-B₁B₁)_{w}-(A₁A₁-B₂B₂)ₓ-(A₂A₂-B₁B₁)_{y}-(A₂A₂-B₂B₂)_{z}, where w, x, y, and z are determined by the relative incorporation of A₁A₁-B₁B₁, A₁A₁-B₂B₂, A₂A₂-B₁B₁, and A₂A₂-B₂B₂ into the polymer backbone. More than two di-acid co-monomers and/or more than two diamine co-monomers can also be used. Therefore, one or more diamine monomers can be polymerized with one or more di-acids, and the general form of the polymer is determined by varying the amount and types of monomers used.

There are many examples of monomers that can be used to make the aerogel polymer compositions containing polyamic amide polymer of the present invention. In some embodiments, the diamine monomer is a substituted or unsubstituted aromatic diamine, a substituted or unsubstituted alkyldiamine, or a diamine that can include both aromatic and alkyl functional groups. A non-limiting list of possible diamine monomers comprises 4,4′-oxydianiline (ODA), 3,4′-oxydianiline, 3,3′-oxydianiline, p-phenylenediamine, m-phenylenediamine, o-phenylenediamine, diaminobenzanilide, 3,5-diaminobenzoic acid, 3,3′-diaminodiphenylsulfone, 4,4′-diaminodiphenyl sulfones, 1,3-bis-(4-aminophenoxy)benzene, 1,3-bis-(3-aminophenoxy)benzene, 1,4-bis-(4-aminophenoxy)benzene, 1,4-bis-(3-aminophenoxy)benzene, 2,2-bis[4-(4-aminophenoxy)phenyl]-hexafluoropropane, 2,2-bis(3-aminophenyl)-1,1,1,3,3,3-hexafluoropropane, 4,4′-isopropylidenedianiline, 1-(4-aminophenoxy)-3-(3-aminophenoxy)benzene, 1-(4-aminophenoxy)-4-(3-aminophenoxy)benzene, bis-[4-(4-aminophenoxy)phenyl]sulfones, 2,2-bis[4-(3-aminophenoxy)phenyl]sulfones, bis(4-[4-aminophenoxy]phenyl)ether, 2,2′-bis-(4-aminophenyl)-hexafluoropropane (6F-diamine), 2,2′-bis-(4-phenoxyaniline)isopropylidene, meta-phenylenediamine, para-phenylenediamine, 1,2-diaminobenzene, 4,4′-diaminodiphenylmethane, 2,2-bis(4-aminophenyl)propane, 4,4′diaminodiphenyl propane, 4,4′-diaminodiphenyl sulfide, 4,4′-diaminodiphenylsulfone, 3,4′diaminodiphenyl ether, 4,4′-diaminodiphenyl ether, 2,6-diaminopyridine, bis(3-aminophenyl)diethyl silane, 4,4′-diaminodiphenyl diethyl silane, benzidine, dichlorobenzidine, 3,3′-dimethoxybenzidine, 4,4′-diaminobenzophenone, N,N-bis(4-aminophenyl)-n-butylamine, N,N-bis(4-aminophenyl)methylamine, 1,5-diaminonaphthalene, 3,3′-dimethyl-4,4′-diaminobiphenyl, 4-aminophenyl-3-aminobenzoate, N,N-bis(4-aminophenyl)aniline, bis(p-beta-amino-t-butylphenyl)ether, p-bis-2-(2-methyl-4-aminopentyl)benzene, p-bis(1,1-dimethyl-5-aminopentyl)benzene, 1,3-bis(4-aminophenoxy)benzene, m-xylenediamine, p-xylenediamine, 4,4′-diaminodiphenyl ether phosphine oxide, 4,4′-diaminodiphenyl N-methyl amine, 4,4′-diaminodiphenyl N-phenyl amine, amino-terminal polydimethylsiloxanes, amino-terminal polypropyleneoxides, amino-terminal polybutyleneoxides, 4,4′-Methylenebis(2-methylcyclohexylamine), 1,2-diaminoethane, 1,3-diaminopropane, 1,4-diaminobutane, 1,5-diaminopentane, 1,6-diaminohexane, 1,7-diaminoheptane, 1,8-diaminooctane, 1,9-diaminononane, 1,10-diaminodecane, and 4,4′-methylenebisbenzeneamine, 2,2′-dimethylbenzidine, (also known as 4,4′-diamino-2,2′-dimethylbiphenyl (DMB)), bisaniline-p-xylidene, 4,4′-bis(4-aminophenoxy)biphenyl, 3,3′-bis(4 aminophenoxy)biphenyl, 4,4′-(1,4-phenylenediisopropylidene)bis aniline, and 4,4′-(1,3-phenylenediisopropylidene)bisaniline, or combinations thereof. In a specified embodiment, the diamine monomer is ODA, 2,2′-dimethylbenzidine, or both.

A non-limiting list of possible dianhydride (“diacid”) monomers includes hydroquinone dianhydride, 3,3′,4,4′-biphenyltetracarboxylic dianhydride (BPDA), pyromellitic dianhydride (PMDA), 3,3′,4,4′-benzophenonetetracarboxylic dianhydride, 4,4′-oxydiphthalic anhydride, 3,3′,4,4′-diphenylsulfonetetracarboxylic dianhydride, 4,4′-(4,4′-isopropylidenediphenoxy)bis(phthalic anhydride), 2,2-bis(3,4-dicarboxyphenyl)propane dianhydride, 4,4′-(hexafluoroisopropylidene)diphthalic anhydride, bis(3,4-dicarboxyphenyl) sulfoxide dianhydride, polysiloxane-containing dianhydride, 2,2′,3,3′-biphenyltetracarboxylic dianhydride, 2,3,2′,3′-benzophenonetetraearboxylic dianhydride, naphthalene-2,3,6,7-tetracarboxylic dianhydride, naphthalene-1,4,5,8-tetracarboxylie dianhydride, 4,4′-oxydiphthalic dianhydride, 3,3′,4,4′-biphenylsulfone tetracarboxylic dianhydride, 3,4,9,10-perylene tetracarboxylic dianhydride, bis(3,4-dicarboxyphenyl)sulfide dianhydride, bis(3,4-dicarboxyphenyl)methane dianhydride, 2,2-bis(3,4-dicarboxyphenyl)propane dianhydride, 2,2-bis(3,4-dicarboxyphenyl)hexafluoropropane, 2,6-dichloronaphthalene-1,4,5,8-tetracarboxylic dianhydride, 2,7-dichloronapthalene-1,4,5,8-tetracarboxylic dianhydride, 2,3,6,7-tetrachloronaphthalene-1,4,5,8-tetracarboxylic dianhydride, phenanthrene, 8,9,10-tetracarboxylic dianhydride, pyrazine-2,3,5,6-tetracarboxylic dianhydride, benzene-1,2,3,4-tetracarboxylic dianhydride, and thiophene-2,3,4,5-tetracarboxylic dianhydride. In a specific embodiment, the dianhydride monomer is BPDA, PMDA, or both.

In some aspects, the molar ratio of anhydride to total diamine is from 0.4:1 to 1.6:1, 0.5:1 to 1.5:1, 0.6:1 to 1.4:1, 0.7:1 to 1.3:1, or specifically from 0.8:1 to 1.2:1. In further aspects, the molar ratio of dianhydride to multifunctional amine (e.g., triamine) is 2:1 to 140:1, 3:1 to 130:1, 4:1 to 120:1, 5:1 to 110:1, 6:1 to 100:1, 7:1 to 90:1, or specifically from 8:1 to 80:1. Mono-anhydride groups can also be used. Non-limiting examples of mono-anhydride groups include 4-amino-1,8-naphthalic anhydride, endo-bicyclo[2.2.2]oct-5-ene-2,3-dicarboxylic anhydride, citraconic anhydride, trans-1,2-cyclohexanedicarboxylic anhydride, 3,6-dichlorophthalic anhydride, 4,5-dichlorophthalic anhydride, tetrachlorophthalic anhydride 3,6-difluorophthalic anhydride, 4,5-difluorophthalic anhydride, tetrafluorophthalic anhydride, maleic anhydride, 1-cyclopentene-1,2-dicarboxylic anhydride, 2,2-dimethylglutaric anhydride 3,3-dimethylglutaric anhydride, 2,3-dimethylmaleic anhydride, 2,2-dimethylsuccinic anhydride, 2,3-diphenylmaleic anhydride, phthalic anhydride, 3-methylglutaric anhydride, methylsuccinic anhydride, 3-nitrophthalic anhydride, 4-nitrophthalic anhydride, 2,3-pyrazinedicarboxylic anhydride, or 3,4-pyridinedicarboxylic anhydride. Specifically, the mono-anhydride group can be phthalic anhydride.

In another embodiment, the polymer compositions used to prepare the aerogels of the present invention include multifunctional amine monomers with at least three primary amine functionalities. The multifunctional amine may be a substituted or unsubstituted aliphatic multifunctional amine, a substituted or unsubstituted aromatic multifunctional amine, or a multifunctional amine that includes a combination of an aliphatic and two aromatic groups, or a combination of an aromatic and two aliphatic groups. A non-limiting list of possible multifunctional amines include propane-1,2,3-triamine, 2-aminomethylpropane-1,3-diamine, 3-(2-aminoethyl)pentane-1,5-diamine, bis(hexamethylene)triamine, N′,N′-bis(2-aminoethyl)ethane-1,2-diamine, N′,N′-bis(3-aminopropyl)propane-1,3-diamine, 4-(3-aminopropyl)heptane-1,7-diamine, N′,N′-bis(6-aminohexyl)hexane-1,6-diamine, benzene-1,3,5-triamine, cyclohexane-1,3,5-triamine, melamine, N-2-dimethyl-1,2,3-propanetriamine, diethylenetriamine, 1-methyl or 1-ethyl or 1-propyl or 1-benzyl-substituted diethylenetriamine, 1,2-dibenzyldiethylenetriamine, lauryldiethylenetriamine, N-(2-hydroxypropyl)diethylenetriamine, N,N-bis(1-methylheptyl)-N-2-dimethyl-1,2,3-propanetriamine, 2,4,6-tris(4-(4-aminophenoxy)phenyl)pyridine, N,N-dibutyl-N-2-dimethyl-1,2,3-propanetriamine, 4,4′-(2-(4-aminobenzyl)propane-1,3-diyl)dianiline, 4-((bis(4-aminobenzyl)amino)methyl)aniline, 4-(2-(bis(4-aminophenethyl)amino)ethyl)aniline, 4,4′-(3-(4-aminophenethyl)pentane-1,5-diyl)dianiline, 1,3,5-tris(4-aminophenoxy)benzene (TAPOB), 4,4′,4″-methanetriyltrianiline, N,N,N′,N′-Tetrakis(4-aminophenyl)-1,4-phenylenediamine, a polyoxypropylenetriamine, octa(aminophenyl)polyhedral oligomeric silsesquioxane, or combinations thereof. A specific example of a polyoxypropylenetriamine is JEFFAMINE® T-403 from Huntsman Corporation, The Woodlands, Tex. USA. In a specific embodiment, the aromatic multifunctional amine may be 1,3,5-tris(4-aminophenoxy)benzene or 4,4′,4″-methanetriyltrianiline. In some embodiments, the multifunctional amine includes three primary amine groups and one or more secondary and/or tertiary amine groups, for example, N′,N′-bis(4-aminophenyl)benzene-1,4-diamine.

Non-limiting examples of capping agents or groups include amines, maleimides, nadimides, acetylene, biphenylenes, norbornenes, cycloalkyls, and N-propargyl and specifically those derived from reagents including 5-norbornene-2,3-dicarboxylic anhydride (nadic anhydride, NA), methyl-nadic anhydride, hexachloro-nadic anhydride, cis-4-cyclohexene-1,2-dicarboxylic anhydride, 4-amino-N-propargylphthalimide, 4-ethynylphthalic anhydride, and maleic anhydride.

The characteristics or properties of the final polymer are significantly impacted by the choice of monomers which are used to produce the polymer. Factors to be considered when selecting monomers include the properties of the final polymer, such as the flexibility, thermal stability, coefficient of thermal expansion (CTE), coefficient of hydroscopic expansion (CHE) and any other properties specifically desired, as well as cost. Often, certain important properties of a polymer for a particular use can be identified. Other properties of the polymer may be less significant, or may have a wide range of acceptable values; so many different monomer combinations could be used.

In some instances, the backbone of the polymer can include substituents. The substituents (e.g., oligomers, functional groups, etc.) can be directly bonded to the backbone or linked to the backbone through a linking group (e.g., a tether or a flexible tether). In other embodiments, a compound or particles can be incorporated (e.g., blended and/or encapsulated) into the polyimide structure without being covalently bound to the polyimide structure. In some instances, the incorporation of the compound or particles can be performed during the polyamic reaction process. In some instances, particles can aggregate, thereby producing polyimides having domains with different concentrations of the non-covalently bound compounds or particles.

Specific properties of a polyimide can be influenced by incorporating certain compounds into the polyimide. The selection of monomers is one way to influence specific properties. Another way to influence properties is to add a compound or property modifying moiety to the polyimide.

### C. Preparation of Polymeric Aerogels

Polymeric aerogel films that can be used in the context of the present invention are commercially available. Non-limiting examples of such films include the Blueshift AeroZero® rolled thin film (available from Blueshift Materials, Inc. (Spencer, Mass.) and Airloy® films (available from Aerogel Technologies, LLC), with the Blueshift AeroZero® rolled thin film being preferred in some aspects. Polymeric aerogel particles that can be used in the context of the present invention are commercially available, Non-limiting examples of such particles include the Blueshift AeroZero® particles (available from Blueshift Materials, Inc. (Spencer, Mass.), Sumteq Thermoplastic Aerogel Particles (can be purchased from Aerogel Technologies, LLC, Boston, Mass.), and Aerogelex Biopolymer Aerogel Particles (can be purchased from Aerogel Technologies, LLC, Boston, Mass.), with the Blueshift AeroZero® particles being preferred in some aspects.

Further, and in addition to the processes discussed below, polymeric aerogels (films, stock shapes or monoliths, particles, etc.) can be made using the methodology described in International Patent Application Publication Nos. WO 2014/189560 to Rodman et al., 2017/07888 to Sakaguchi et al., 2018/078512 to Yang et al. 2018/140804 to Sakaguchi et al., 2019/006184 to Irvin et al., International Patent Application No. PCT/US2019/029191 to Ejaz et al., U.S. Patent Application Publication No. 2017/0121483 to Poe et al., and/or U.S. Pat. No. 9,963,571 to Sakaguchi et al., all of which are incorporated herein by reference in their entirety.

The following provides non-limiting processes that can be used to make the polymeric aerogel matrices used in the air-permeable filter material of the present invention. These processes can include: 1) preparation of the polymer gel, 2) optional solvent exchange, 3) drying of the polymeric solution to form the aerogel; 4) attaching a polymeric aerogel film on a substrate; and 5) producing polymeric aerogel particles.

1. Formation of a Polymer Gel

The first stage in the synthesis of an aerogel can be the synthesis of a polymerized gel. For example, if a polyimide aerogel is desired, at least one acid monomer can be reacted with at least one diamino monomer in a reaction solvent to form a polyamic acid. As discussed above, numerous acid monomers and diamino monomers may be used to synthesize the polyamic acid. In one aspect, the polyamic acid is contacted with an imidization catalyst in the presence of a chemical dehydrating agent to form a polymerized polyimide gel via an imidization reaction. “Imidization” is defined as the conversion of a polyimide precursor into an imide. Any imidization catalyst suitable for driving the conversion of polyimide precursor to the polyimide state is suitable. Non-limiting examples of chemical imidization catalysts include pyridine, methylpyridines, quinoline, isoquinoline, 1,8-diazabicyclo[5.4.0]undec-7-ene (DBU), triethylenediamine, lutidine, N-methylmorpholine, triethylamine, tripropylamine, tributylamine, other trialkylamines, 2-methyl imidazole, 2-ethyl-4-methylimidazole, imidazole, other imidazoles, and combinations thereof. Any dehydrating agent suitable for use in formation of an imide ring from an amic acid precursor is suitable for use in the methods of the present invention. Preferred dehydrating agents comprise at least one compound selected from the group consisting of acetic anhydride, propionic anhydride, n-butyric anhydride, benzoic, anhydride, trifluoroacetic anhydride, phosphorus trichloride, and dicyclohexylcarbodiimide.

In one aspect of the current invention, one or more diamino monomers and one or more multifunctional amine monomers are premixed in one or more solvents and then treated with one or more dianhydrides (e.g., di-acid monomers) that are added in sequentially smaller amounts at pre-defined time increments while monitoring the viscosity. The desired viscosity of the polymerized solution can range from 50 to 20,000 cP or specifically 500 to 5,000 cP. By performing the reaction using incremental addition of dianhydride while monitoring viscosity, a non-crosslinked aerogel can be prepared. For instance, a triamine monomer (23 equiv.) can be added to the solvent to give a 0.0081 molar solution. To the solution a first diamine monomer (280 equiv.) can be added, followed by second diamine monomer (280 equiv.). Next a dianhydride (552 total equiv.) can be added in sequentially smaller amounts at pre-defined time increments while monitoring the viscosity. The dianhydride can be added until the viscosity reaches 1,000 to 1,500 cP. For example, a first portion of dianhydride can be added, the reaction can be stirred (e.g., for 20 minutes), a second portion of dianhydride can be added, and a sample of the reaction mixture was then analyzed for viscosity. After stirring for additional time (e.g., for 20 minutes), a third portion of dianhydride can be added, and a sample can be taken for analysis of viscosity. After further stirring for a desired period of time (e.g., 10 hours to 12 hours), a mono-anhydride (96 equiv.) can be added. After having reached the target viscosity, the reaction mixture can be stirred for a desired period of time (e.g., 10 hours to 12 hours) or the reaction is deemed completed.

The reaction temperature for the gel formation can be determined by routine experimentation depending on the starting materials. In a preferred embodiment, the temperature range can be greater than, equal to, or between any two of 15° C., 20° C., 30° C., 35° C., 40° C., and 45° C. After a desired amount of time (e.g., about 2 hours), the product can be isolated (e.g., filtered), after which a nitrogen containing hydrocarbon (828 equiv.) and dehydration agent (1214 equiv.) can be added. The addition of the nitrogen containing hydrocarbon and/or dehydration agent can occur at any temperature. In some embodiments, the nitrogen containing hydrocarbon and/or dehydration agent is added to the solution at 20° C. to 28° C. (e.g., room temperature) stirred for a desired amount of time at room temperature. In some instances, after addition of nitrogen containing hydrocarbon and/or dehydration agent, the solution temperature is raised up to 150° C.

The reaction solvent can include dimethylsulfoxide (DMSO), diethylsulfoxide, N,N-dimethylformamide (DMF), N,N-diethylformamide, N,N-dimethylacetamide (DMAc), N,N-diethylacetamide, N-methyl-2-pyrrolidone (NMP), 1-methyl-2-pyrrolidinone, N-cyclohexyl-2-pyrrolidone, 1,13-dimethyl-2-imidazolidinone, diethyleneglycoldimethoxyether, o-dichlorobenzene, phenols, cresols, xylenol, catechol, butyrolactones, hexamethylphosphoramide, and mixtures thereof. The reaction solvent and other reactants can be selected based on the compatibility with the materials and methods applied i.e. if the polymerized polyamic amide gel is to be cast onto a support film, injected into a moldable part, or poured into a shape for further processing into a workpiece. In a specific embodiment, the reaction solvent is DMSO.

While keeping the above in mind, the introduction of macropores into the aerogel polymeric matrix, as well as the amount of such macropores present, can be performed in the manner described above in the Summary of the Invention Section as well as throughout this specification. In one non-limiting manner, the formation of macropores versus smaller mesopores and micropores can be primarily controlled by controlling the polymer/solvent dynamics during gel formation. By doing so, the pore structure can be controlled, and the quantity and volume of macroporous, mesoporous, microporous cells can be controlled. For example, a curing additive that reduces the solubility of the polymers being formed during polymerization step (b), such as 1,4-diazabicyclo[2.2.2]octane, can produce a polymer gel containing a higher number of macropores as compared to another curing additive that improves the resultant polymer solubility, such as triethylamine. In another specific non-limiting example when forming a polyimide aerogel having macropores, increasing the ratio of rigid amines incorporated into the polymer backbone such as p-phenylenediamine (p-PDA) as compared to more flexible diamines such as -ODA, the formation of macropores as compared to smaller mesopores and micropores can be controlled.

The polymer solution may optionally be cast onto a casting sheet covered by a support film for a period of time. Casting can include spin casting, gravure coating, three roll coating, knife over roll coating, slot die extrusion, dip coating, Meyer rod coating, or other techniques. In one embodiment, the casting sheet is a polyethylene terephthalate (PET) casting sheet. After a passage of time, the polymerized reinforced gel is removed from the casting sheet and prepared for the solvent exchange process. In some embodiments, the cast film can be heated in stages to elevated temperatures to remove solvent and convert the amic acid functional groups in the polyamic acid to imides with a cyclodehydration reaction, also called imidization. In some instances, polyamic acids may be converted in solution to polyimides with the addition of the chemical dehydrating agent, catalyst, and/or heat.

In some embodiments, the polyimide polymers can be produced by preparing a polyamic acid polymer in the reaction vessel. The polyamic acid is then formed into a sheet or a film and subsequently processed with catalysts or heat and catalysts to convert the polyamic acid to a polyimide.

2. Optional Solvent Exchange

After the polymer gel is synthesized, it may be desirable in certain instances to conduct a solvent exchange wherein the reaction solvent is exchanged for a more desirable second solvent. Accordingly, in one embodiment, a solvent exchange can be conducted wherein the polymerized gel is placed inside of a pressure vessel and submerged in a mixture comprising the reaction solvent and the second solvent. Then, a high pressure atmosphere is created inside of the pressure vessel thereby forcing the second solvent into the polymerized gel and displacing a portion of the reaction solvent. Alternatively, the solvent exchange step may be conducted without the use of a high pressure environment. It may be necessary to conduct a plurality of rounds of solvent exchange. In some embodiments, solvent exchange is not necessary.

The time necessary to conduct the solvent exchange will vary depending upon the type of polymer undergoing the exchange as well as the reaction solvent and second solvent being used. In one embodiment, each solvent exchange can range from 1 to 168 hours or any period time there between including 2, 3, 4, 5, 6, 7, 8, 9, 10, 11, 12, 13, 14, 15, 16, 17, 18, 19, 20, 21, 22, or 23, 24, 25, 50, 75, 100, 125, 150, 155, 160, 165, 166, 167, or 168 hours. In another embodiment, each solvent exchange can take approximately 1 to 60 minutes, or about 30 minutes. Exemplary second solvents include methanol, ethanol, 1-propanol, 2-propanol, 1-butanol, 2-butanol, isobutanol, tert-butanol, 3-methyl-2-butanol, 3,3-dimethyl-2-butanol, 2-pentanol, 3-pentanol, 2,2-dimethylpropan-1-ol, cyclohexanol, diethylene glycol, cyclohexanone, acetone, acetyl acetone, 1,4-dioxane, diethyl ether, dichloromethane, trichloroethylene, chloroform, carbon tetrachloride, water, and mixtures thereof. In certain non-limiting embodiments, the second solvent can have a suitable freezing point for performing supercritical or subcritical drying steps. For example tert-butyl alcohol has a freezing point of 25.5° C. and water has a freezing point of 0° C. under one atmosphere of pressure. Alternatively, and as discussed below, however, the drying can be performed without the use of supercritical or subcritical drying steps, such as by evaporative drying techniques.

The temperature and pressure used in the solvent exchange process may be varied. The duration of the solvent exchange process can be adjusted by performing the solvent exchange at a varying temperatures or atmospheric pressures, or both, provided that the pressure and temperature inside the pressure vessel does not cause either the first solvent or the second solvent to leave the liquid phase and become gaseous phase, vapor phase, solid phase, or supercritical fluid. Generally, higher pressures and/or temperatures decrease the amount of time required to perform the solvent exchange, and lower temperatures and/or pressures increase the amount of time required to perform the solvent exchange.

3. Cooling and Drying

In one embodiment after solvent exchange, the polymerized gel can be exposed to supercritical drying. In this instance the solvent in the gel can be removed by supercritical CO₂ extraction.

In another embodiment after solvent exchange, the polymerized reinforced gel can be exposed to subcritical drying. In this instance, the gel can be cooled below the freezing point of the second solvent and subjected to a freeze drying or lyophilization process to produce the aerogel. For example, if the second solvent is water, then the polymerized gel is cooled to below 0° C. After cooling, the polymerized gel can be subjected to a vacuum for a period of time to allow sublimation of the second solvent

In still another embodiment, after solvent exchange, the polymerized gel can be exposed to subcritical drying with optional heating after the majority of the second solvent has been removed through sublimation. In this instance the partially dried gel material is heated to a temperature near or above the boiling point of the second solvent for a period of time. The period of time can range from a few hours to several days, although a typical period of time is approximately 4 hours. During the sublimation process, a portion of the second solvent present in the polymerized gel has been removed, leaving a gel that can have macropores, mesopores, or micropores, or any combination thereof or all of such pore sizes. After the sublimation process is complete, or nearly complete, the aerogel has been formed.

In yet another embodiment after solvent exchange, the polymerized gel can be dried under ambient conditions, for example, by removing the solvent under a stream of gas (e.g., air, anhydrous gas, inert gas (e.g., nitrogen (N₂) gas), etc.). Still further, passive drying techniques can be used such as simply exposing the gel to ambient conditions without the use of a gaseous stream.

Once cooled or dried, the films and stock shapes can be configured for use in the air filter materials 1 of the present invention. For example, the films or stock shapes can be processed into desired shapes (e.g., by cutting or grinding) such as square shapes, rectangular shapes, circular shapes, triangular shapes, irregular shapes, random shapes, etc. Also, and as discussed above, the films or stock shapes can be affixed to a support material such as with an adhesive. In alternative embodiments where an adhesive may not be desired, a support material can be incorporated into the matrix of the polymeric aerogel, which is discussed below. Alternatively, and also discussed below, the polymeric aerogels can be made into particulate form.

4. Incorporation of a Support Material into the Matrix of the Polymeric Aerogel

In addition to the methods discussed above with respect to the use of adhesives for attaching a polymeric aerogel to a support material, an optional embodiment of the present invention can include incorporation of the support material into the polymeric matrix to create a reinforced polymeric aerogel without the use of adhesives. Notably, during manufacture of a non-reinforced polymer aerogel a reinforcing support film can be used as a carrier to support the gelled film during processing. During rewinding, the gelled film can be irreversibly pressed into the carrier film. Pressing the gelled film into the carrier film can provide substantial durability improvement. In another instance, during the above-mentioned solvent casting step, the polymer solution can be cast into a reinforcement or support material.

The substrate selection and direct casting can allow optimization of (e.g., minimization) of the thickness of the resulting reinforced aerogel material. This process can also be extended to the production of fiber reinforced polymer aerogels—internally reinforced polyimide aerogels are provided as an example. The process can include: (a) forming a polyamic acid solution from a mixture of dianhydride and diamine monomers in a polar solvent such as DMSO, DMAc, NMP, or DMF; (b) contacting the polyamic acid solution with chemical curing agents listed above and a chemical dehydrating agent to initiate chemical imidization; (c) casting the polyamic acid solution onto a fibrous support prior to gelation and allow it to permeate it; (d) allowing the catalyzed polyamic acid solution to gel around, and into, the fibrous support during chemical imidization; (e) optionally performing a solvent exchange, which can facilitate drying; and (f) removal of the transient liquid phase contained within the gel with supercritical, subcritical, or ambient drying to give an internally reinforced aerogel. The polyimide aerogels can be produced from aromatic dianhydride and diamine monomers, such as aromatic diamines or a mixture of at least one aromatic diamine monomer and at least one aliphatic diamine monomer. The resulting polyimide aerogel can be optimized to possess low density, narrow pore size distribution and good mechanical strength. The polyimide aerogel can also be optimized to include mesopores, micropores, or macropores, or any combination thereof or all such pore sizes.

The preparation of polyimide wet gels can be a two-step procedure: (a) formation of the polyamic acid solution from a mixture of dianhydride and diamine in a polar solvent such as DMAc, NMP, DMF, or DMSO; and (b) catalyzed cyclization with chemical catalyzing agents to form a polyimide. In some embodiments, at least 30 minutes mixing at room temperature can be performed to allow for formation of the polyimide polymer and yielding of stable, robust wet gels. Gelation conditions depend on several factors, including the prepared density of the solution and the temperature of the heating oven. Higher concentration solutions can gel faster than lower density solutions. Once the system has reached the gelled state, the gels are optionally rinsed repeatedly with acetone, ethanol, or the like. Rinsing occurs at least three times prior to drying, and serves to remove residual solvent and unreacted monomers. CO₂ can then be used in techniques known to those in the art for wet solvent extraction to create the aerogel structure. Other techniques for preparing and optimizing polyimide aerogels can be used and are known in the art.

The reinforced macroporously structured aerogels of the present invention can be any width or length and can be in the form of defined geometry (e.g., a square or circular patch or any other stock shape), or in the form of a sheet or roll. In some instances, the internally reinforced aerogels can have a width up to 6 meters and a length of up to 10 meters, or from 0.01 to 6 meters, 0.5 to 5 meters, 1 to 4 meters, or any range in between, and a length of 1 to 10,000 meters, 5 to 1,000 meters, 10 to 100 meters or any range there between. The width of the composite can be 0.01, 0.05, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1.0, 1.5, 2.0, 2.5, 3.0, 3.5, 4.0, 4.5, 5.0, 5.5, 6.0 meters, including any value there between. The length of the internally reinforced aerogels can be 0.1, 1, 10, 100, 500, 1000, 1500, 2000, 2500, 3000, 3500, 4000, 4500, 5000, 5500, 6000, 6500, 7000, 7500, 8000, 8500, 9000, 9500, 10000 meters and include any value there between. In certain aspect the length of the internally reinforced aerogel can be 1000 meters, and 1.5 meters, respectively, in width. In a further embodiment the internally reinforced aerogel is 100 feet (30.5 meters) in length and 40 inches (1.0 meter) wide.

In certain embodiments the internally reinforced aerogel includes a non-woven support at least partially or fully embedded or incorporated in a polymeric aerogel.

The support can be comprised of a plurality of fibers. The fibers can be unidirectionally or omnidirectionally oriented. The support can include, by volume, at least 0.1 to 50% of the internally reinforced aerogel. The support can be in the form of a plurality of fibers, a film or layer of fibers, fiber containing films or layers, or a support film or layer comprising two or more fiber layers pressed together to form the support. The support can include cellulose fibers, glass fibers, carbon fibers, aramid fibers, thermoplastic fibers (e.g., polyethylene fibers, polyester, nylon, etc.), thermoset fibers (e.g., rayon, polyurethane, and the like), ceramic fibers, basalt fibers, rock wool, or steel fibers, or mixtures thereof. The fibers can have an average filament cross sectional area of 7 μm² to 800 μm², which equates to an average diameter of 3 to 30 microns for circular fibers. Bundles of various kinds of fibers can be used depending on the use intended for the internally reinforced aerogel. For example, the bundles may be of carbon fibers or ceramic fibers, or of fibers that are precursors of carbon or ceramic, glass fibers, aramid fibers, or a mixture of different kinds of fiber. Bundles can include any number of fibers. For example, a bundle can include 400, 750, 800, 1375, 1000, 1500, 3000, 6000, 12000, 24000, 50000, or 60000 filaments. The fibers can have a filament diameter of 5 to 24 microns, 10 to 20 microns, or 12 to 15 microns or any range there between, or 5, 6, 7, 8, 9, 10, 11, 12, 13, 14, 15, 16, 17, 18, 19, 20, 21, 22, 23, 24 microns or any value there between. The fibers in a bundle of fibers can have an average filament cross sectional area of 7 μm² to 800 μm², which equates to an average diameter of 3 to 30 microns for circular fibers. Cellulose and paper supports can be obtained from Hirose Paper Mfg Co (Kochi, Japan) or Hirose Paper North America (Macon, Ga., USA).

Thermoplastic and thermoset fibers can include thermoplastic and/or thermoset polymers. Non-limiting examples of thermoplastic polymers include polyethylene terephthalate (PET), a polycarbonate (PC) family of polymers, polybutylene terephthalate (PBT), poly(1,4-cyclohexylidene cyclohexane-1,4-dicarboxylate) (PCCD), glycol modified polycyclohexyl terephthalate (PCTG), poly(phenylene oxide) (PPO), polypropylene (PP), polyethylene (PE), polyvinyl chloride (PVC), polystyrene (PS), polymethyl methacrylate (PMMA), polyethyleneimine or polyetherimide (PEI) and their derivatives, thermoplastic elastomer (TPE), terephthalic acid (TPA) elastomers, poly(cyclohexanedimethylene terephthalate) (PCT), polyethylene naphthalate (PEN), polyamide (PA), polysulfone sulfonate (PSS), sulfonates of polysulfones, polyether ether ketone (PEEK), polyether ketone (PEKK), acrylonitrile butyldiene styrene (ABS), polyphenylene sulfide (PPS), co-polymers thereof, polyesters or derivatives thereof, polyamides or derivatives thereof (e.g., nylon), or blends thereof.

Non-limiting examples of thermoset polymers include unsaturated polyester resins, polyurethanes, polyoxybenzylmethylenglycolanhydride (e.g., Bakelite), urea-formaldehyde, diallyl-phthalate, epoxy resin, epoxy vinylesters, polyimides, cyanate esters of polycyanurates, dicyclopentadiene, phenolics, benzoxazines, co-polymers thereof, or blends thereof.

In other aspects, the internally reinforced aerogel can includes two or more layers of a support. In certain instances, a support can include two unidirectional supports in contact with each other and arranged such that the unidirectional fibers are oriented in different directions to each other. In other instances, the support can comprises two or more layers of a support having omnidirectional fibers.

The support can be positioned at least partially or fully inside a polymeric aerogel, forming an internal support and an external aerogel. As used herein any support that is at least partially permeated with aerogel material can be partially internalized. The width and length of the aerogel can be substantially similar to the width and length of the internal or partially internalized support.

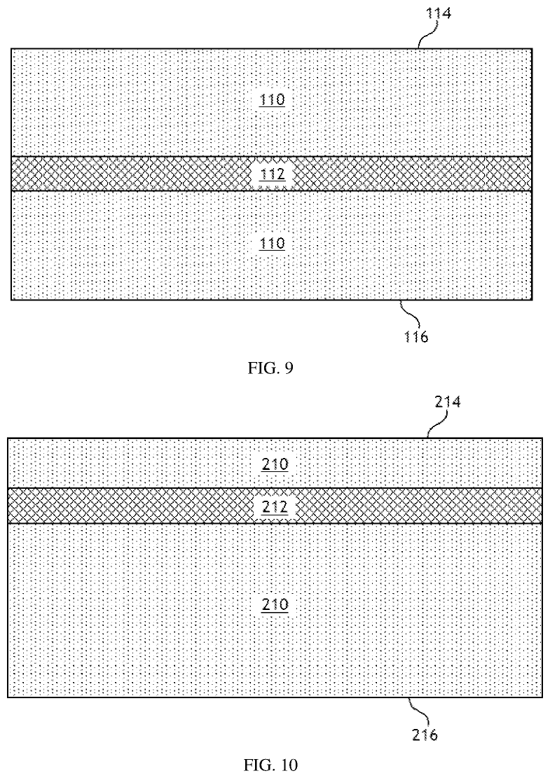

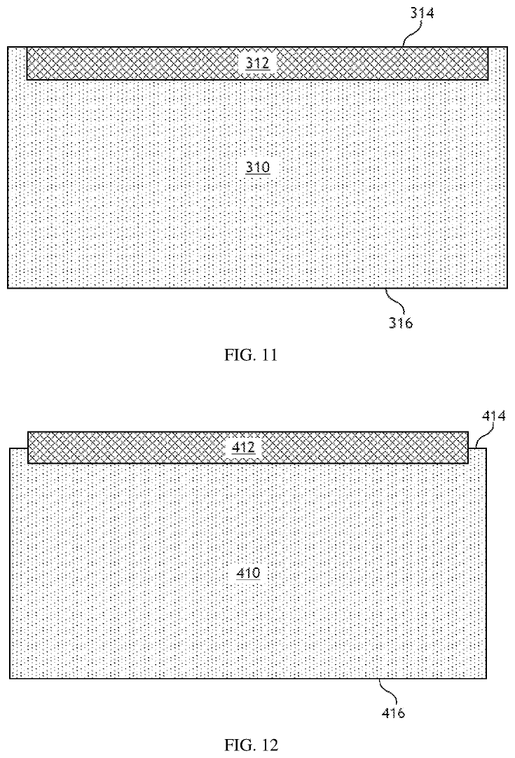

FIG. 9 illustrates an internally reinforced aerogel having internal support 112 positioned at about the midline of aerogel 110. Support 112 is approximately equidistant from top edge 114 and bottom edge 116. FIG. 10 illustrates and embodiment where internal support 212 is in an offset position within aerogel 210. Support 212 being closer to one edge (in the illustrated case top edge 214) than the other edge (bottom edge 216). In other embodiments support 212 can be positioned closer to the bottom edge. FIG. 11 illustrates an embodiment where the outer edge of support 312 is positioned along the top edge 314 of aerogel 310. In other embodiments support 312 can be positioned along bottom edge 316. FIG. 12 illustrates another embodiment where support 412 is partially incorporated into aerogel 410. In this embodiments a portion of the support is above or outside top edge 414. In other embodiments the position of the support can be at bottom edge 416.

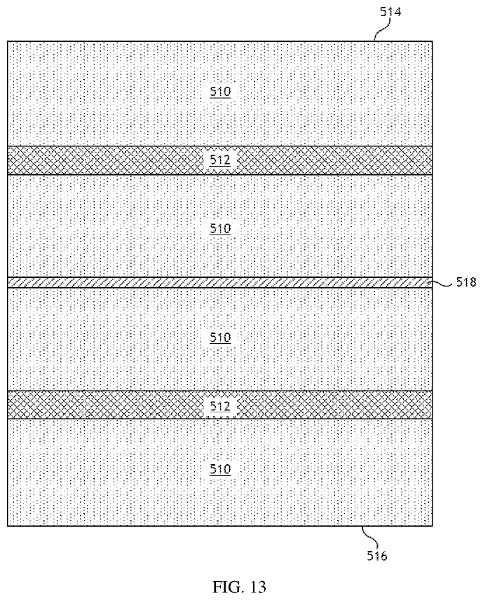

In certain embodiments, a reinforced aerogel laminate can be constructed having 2, 3, 4, 5 or more reinforced aerogel layers (See FIG. 13). FIG. 13 shows a two-layer laminate. In this example each layer is configured as shown in FIG. 9; however, any number of reinforced aerogel configurations can be used and in any combination. Each of the reinforced aerogel layer depicted in FIG. 13 include aerogel 510 and support 512. The layers can be adhered to each through an aerogel/aerogel interface or by an adhesive 518. The laminate having top edge 514 and a bottom edge 516.

The cross-sectional thickness of the internally reinforced aerogel measured from top most edge to bottom most edge can be any value. In some embodiments the cross-sectional thickness is between 0.02 to 0.5 mm, including all values and ranges there between. The support can be positioned in the aerogel so that about 0, 0.001, 0.01, 0.025, 0.05, 0.075, 0.1, 0.125, 0.15, 0.175, 0.2, 0.225, 0.25, 0.275, 0.3, 0.325, 0.35, 0.375, 0.4 mm of the aerogel is above the support. In certain instances, the support can be approximately within about 0.5 mils (0.013 mm) of the aerogel midline. In a further aspect about 0.1 to 0.5 mil (0.0025 to 0.013 mm) of support extends beyond one of the aerogel edges with a portion of the support being embedded or incorporated in the aerogel.

The present invention will be described in greater detail by way of specific examples. The following examples are offered for illustrative purposes only, and are not intended to limit the invention in any manner. Those of skill in the art will readily recognize a variety of noncritical parameters, which can be changed or modified to yield essentially the same results.

5. Formation of Polymeric Aerogel Particles

Once the aerogel films or stock/monolithic shapes are made, the films or shapes can then be milled, chopped, or machined into particles. The aerogel particles can be any size. In some embodiments, the aerogel particle size can be 1 μm to 500 μm, or at least, equal to, or between any two of 1, 2, 3, 4, 5, 10 20, 30, 40, 50, 60, 70, 80, 90, 100, 150, 200, 250, 300, 350, 400, 450 and 500 μm. In some embodiments, the particle size distribution can be single-modal or multi-modal (e.g., bimodal, trimodal, etc.). In certain embodiments, the particle size distribution is bimodal with one mode being between 10 and 100 μm and the other mode being between 150 and 300 μm. Alternatively, the aerogel particles can be purchased (see above non-limiting commercially available options).

### EXAMPLES

The present invention will be described in greater detail by way of specific examples. The following examples are offered for illustrative purposes only, and are not intended to limit the invention in any manner. Those of skill in the art will readily recognize a variety of noncritical parameters, which can be changed or modified to yield essentially the same results.

Table 2 lists the acronyms for the compounds used in the following Examples.

TABLE 2

| Acronym | Name |
|---|---|
| BPDA | 3,3′,4,4′-biphenyltetracarboxylic |
| | dianhydride |
| DMB | 4,4′-Diamino-2,2′-dimethylbiphenyl |
| DMSO | Dimethylsulfoxide |
| PA | Phthalic anhydride |
| PMDA | Pyromellitic dianhydride |
| ODA | 4,4′-Oxydianiline |
| TAPOB | 1,3,5-Tris(4-aminophenoxy) benzene |

Structures of the starting materials are shown below.

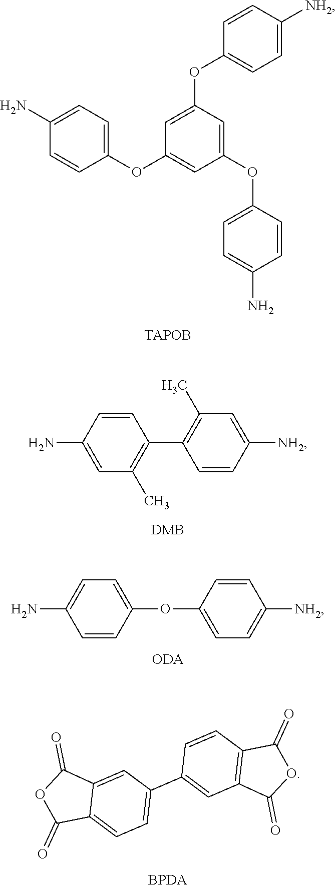

### Example 1

### Preparation of a Highly Branched BPDA/DMB-ODA Polyimide

A reaction vessel with a mechanical stirrer and a water jacket was used. The flow of the water through the reaction vessel jacket was adjusted to maintain temperature in the range of 18-35° C. The reaction vessel was charged with DMSO (108.2 lbs. 49.1 kg), and the mechanical stirrer speed was adjusted to 120-135 rpm. TAPOB (65.13 g) was added to the solvent. To the solution was added DMB (1081.6 g), followed by ODA (1020.2 g). A first portion of BPDA (1438.4 g) was then added. After stirring for 20 minutes, a sample of the reaction mixture was analyzed for viscosity using a Brookfield DV1 viscometer (Brookfield, AMETEK, U.S.A.). A second portion of BPDA (1407.8 g) was added, and the reaction mixture was stirred for 20 additional minutes. A third portion of BPDA (138.62 g) was added, and the reaction mixture was stirred for 20 minutes. A sample of the reaction mixture was analyzed for viscosity. After stirring for 8 hours, PA (86.03 g) was added. The resulting reaction mixture was stirred until no more solids were visible. After 2 hours, the product was removed from the reaction vessel, filtered, and weighed.

### Example 2

### Preparation of a Highly Branched Polyimide Aerogel Monolith by Freeze Drying

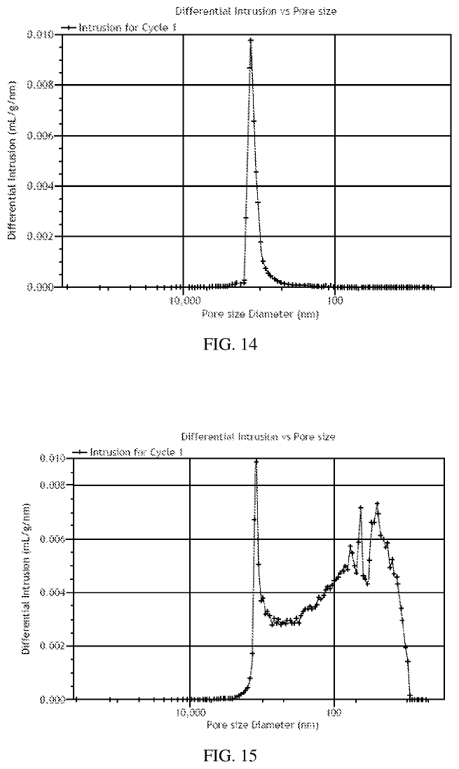

The resin (about 10,000 grams) prepared in Example 1 was mixed with triethylamine (about 219 grams) and acetic anhydride (about 561 grams) for five minutes. After mixing, the resultant solution was poured into a square 15″×15″ mold and left for 48 hours. The gelled shape was removed from the mold, and placed into an acetone bath. After immersion for 24 hours, the acetone bath was exchanged with fresh acetone. The soak and exchange process was repeated five times. After the final exchange, the bath was replaced with tertiary butyl alcohol. After immersion for 24 hours, the tertiary butyl alcohol bath was exchanged for fresh tertiary butyl alcohol. The soak and exchange process was repeated three times The part was subsequently flash frozen and subjected to subcritical drying for 96 hours in at 5° C., followed by drying in vacuum at 50° C. for 48 hours. The final recovered aerogel part had open-cell structure as observed by scanning electron microscopy (SEM) performed on a Phenom Pro Scanning Electron Microscope (Phenom-World, the Netherlands), exhibited a density of 0.22 g/cm³ and porosity of 88.5% as measured according to ASTM D4404-10 with a Micromeritics® AutoPore V 9605 Automatic Mercury Penetrometer (Micromeritics® Instrument Corporation, U.S.A.), a compression modulus of 2.2 MPa as determined by American Standard Testing Method (ASTM) D395-16, and a compression strength at 25% strain of 3.5 MPa as determined by ASTM D395-16. The distribution of pore sizes was measured according to ASTM D4404-10 using a Micromeritics® AutoPore V 9605 Automatic Mercury Penetrometer (Micromeritics® Instrument Corporation, U.S.A.), and the distribution of pore diameters is provided in FIG. 14. From the data it was determined that 100% of the pores were macropores and that the average pore diameter was about 1200 nm, thus confirming that a macroporously shaped aerogel was produced.

### Example 3

### Preparation of a Highly Branched Polyimide Aerogel Monolith by Thermal Drying

The resin (about 10,000 grams) prepared in Example 1 was mixed with triethylamine (about 219 grams) and acetic anhydride (about 561 grams) for five minutes at a temperature of 10-35° C. After mixing, the resultant solution was poured into a square 15″×15″ mold and left for 48 hours. The gelled shape was removed from the mold and placed into an acetone bath. After immersion for 24 hours, the acetone bath was exchanged with fresh acetone. The soak and exchange process was repeated five times. After the final exchange, the part was dried with an ambient (about 20 to 30° C.) drying process to evaporate a majority of the acetone over 48 hours followed by thermal drying at 50° C. for 4 hours, 100° C. for 2 hours, 150° C. for 1 hour, and then 200° C. for 30 minutes. The final recovered aerogel had similar properties as observed in Example 2.

### Example 4

### Preparation of a Highly Branched Polyimide

TAPOB (about 2.86 g) was added to the reaction vessel charged with about 2,523.54 g DMSO as described in Example 1 at a temperature of 18-35° C. To the solution was added a first portion of DMB (about 46.75 g), followed by a first portion of ODA (about 44.09 g). After stirring for about 20 minutes, a first portion of BPDA (about 119.46 g) was added. After stirring for about 20 minutes, TAPOB (about 2.86 g), DMB (about 46.75 g), and ODA (about 44.09 g) were added. After stirring for about 20 minutes, BPDA (about 119.46 g) was added. After stirring for about 20 minutes, TAPOB (about 2.86 g), DMB (about 46.75 g), and ODA (about 44.09 g) were added. After stirring for about 20 minutes, BPDA (about 119.46 g) was added. After stirring for about 8 hours, PA (about 50.12 g) was added. The resulting reaction mixture was stirred until no more solids were visible. After about 2 hours, the product was removed from the reaction vessel, filtered, and weighed.

### Example 5

### Preparation of a Highly Branched Polyimide Aerogel Monolith by Freeze Drying

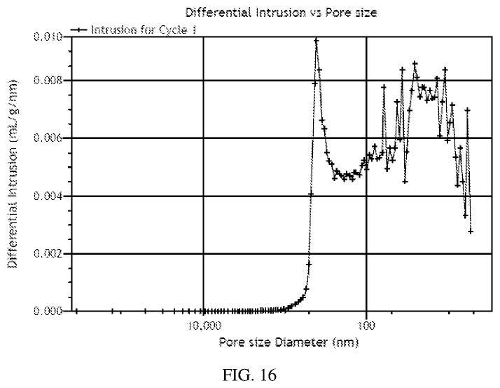

The resin (about 400 grams) prepared in Example 4 was mixed with 2-methylimidazole (about 53.34 grams) for five minutes and then benzoic anhydride (about 161.67 grams) for five minutes at a temperature of 18-35° C. After mixing, the resultant solution was poured into a square 3″×3″ mold and placed in an oven at 75° C. for 30 minutes and then left overnight at room temperature. The gelled shape was removed from the mold, and placed into an acetone bath. After immersion for 24 hours, the acetone bath was exchanged with fresh acetone. The soak and exchange process was repeated five times. After the final exchange, the bath was replaced with tertiary butyl alcohol. After immersion for 24 hours, the tertiary butyl alcohol bath was exchanged for fresh tertiary butyl alcohol. The soak and exchange process was repeated three times The part was subsequently frozen on a shelf freezer, and subjected to subcritical drying for 96 hours in at 5° C., followed by drying in vacuum at 50° C. for 48 hours. The final recovered aerogel part had open-cell structure as observed by scanning electron microscopy (SEM) performed on a Phenom Pro Scanning Electron Microscope (Phenom-World, the Netherlands), exhibited a density of 0.15 g/cm³ and porosity of 92.2% as measured according to ASTM D4404-10 with a Micromeritics® AutoPore V 9605 Automatic Mercury Penetrometer (Micromeritics® Instrument Corporation, U.S.A.). The distribution of pore sizes were measured according to ASTM D4404-10 using a Micromeritics® AutoPore V 9605 Automatic Mercury Penetrometer (Micromeritics® Instrument Corporation, U.S.A.), and the distribution of pore diameters is shown in FIG. 15. From the data, it was determined that the 96.3% of the shaped aerogel's pore volume was made up of pores having an average pore diameter of greater than 50 nm (e.g., from 5 nm to 50 nm), and thus a macroporously structured shaped aerogel was formed.

### Example 6

### Preparation of a Highly Branched Polyimide

TAPOB (about 2.05 g) was added to the reaction vessel charged with about 2,776.57 g DMSO as described in Example 1 at a temperature of 18-35° C. To the solution was added a first portion of DMB (about 33.54 g), followed by a first portion of ODA (about 31.63 g). After stirring for about 20 minutes, a first portion of PMDA (about 67.04 g) was added. After stirring for about 20 minutes, TAPOB (about 2.05 g), DMB (about 33.54 g), and ODA (about 31.63 g) were added. After stirring for about 20 minutes, PMDA (about 67.04 g) was added. After stirring for about 20 minutes, TAPOB (about 2.05 g), DMB (about 33.54 g), and ODA (about 31.63 g) were added. After stirring for about 20 minutes, PMDA (about 67.04 g) was added. After stirring for about 8 hours, PA (about 18.12 g) was added. The resulting reaction mixture was stirred until no more solids were visible. After about 2 hours, the product was removed from the reaction vessel, filtered, and weighed.

### Example 7

### Preparation of a Highly Branched Polyimide Aerogel Monolith by Freeze Drying

The resin (about 400 grams) prepared in Example 6 was mixed with 2-methylimidazole (about 40.38 grams) for five minutes and then benzoic anhydride (about 122.38 grams) for five minutes at a temperature of 18-35° C. After mixing, the resultant solution was poured into a square 3″×3″ mold and placed in an oven at 75° C. for 30 minutes and then left overnight at room temperature. The gelled shape was removed from the mold, and placed into an acetone bath. After immersion for 24 hours, the acetone bath was exchanged with fresh acetone. The soak and exchange process was repeated five times. After the final exchange, the bath was replaced with tertiary butyl alcohol. After immersion for 24 hours, the tertiary butyl alcohol bath was exchanged for fresh tertiary butyl alcohol. The soak and exchange process was repeated three times The part was subsequently frozen on a shelf freezer, and subjected to subcritical drying for 96 hours in at 5° C., followed by drying in vacuum at 50° C. for 48 hours. The final recovered aerogel part had open-cell structure as observed by scanning electron microscopy (SEM) performed on a Phenom Pro Scanning Electron Microscope (Phenom-World, the Netherlands), exhibited a density of 0.23 g/cm³ and porosity of 82.7% as measured according to ASTM D4404-10 with a Micromeritics® AutoPore V 9605 Automatic Mercury Penetrometer (Micromeritics® Instrument Corporation, U.S.A.). The distribution of pore sizes was measured according to ASTM D4404-10 using a Micromeritics® AutoPore V 9605 Automatic Mercury Penetrometer (Micromeritics® Instrument Corporation, U.S.A.), and the distribution of pore diameters is shown in FIG. 16. From the data, it was determined that 90.6% of the shaped macroporously structured aerogel's pore volume was made up of pores having at least an average pore diameter greater than 50 nm.

### Example 8

### Preparation of a Highly Branched Polyamic Film

A reaction vessel with a mechanical stirrer and a water jacket was employed. The flow of the water through the reaction vessel jacket was adjusted to maintain temperature in the range of 20-28° C. The reaction vessel was charged with DMSO (108.2 lbs. 49.1 kg), and the mechanical stirrer speed was adjusted to 120-135 rpm. TAPOB (65.03 g) was added to the solvent. To the solution was added DMB (1,080.96 g), followed by ODA (1,018.73 g). A first portion of BPDA (1,524.71 g) was added. After stirring for 20 minutes, a sample of the reaction mixture was analyzed for viscosity. A second portion of BPDA (1,420.97 g) was added, and the reaction mixture was stirred for 20 additional minutes. A sample of the reaction mixture was analyzed for viscosity. A third portion of BPDA (42.81 g) was added, and the reaction mixture was stirred for 20 additional minutes. A sample of the reaction mixture was analyzed for viscosity. After stirring for 8 hours, PA (77.62 g) was added. The resulting reaction mixture was stirred until no more solid was visible. After 2 hours, the resin was removed from the reaction vessel, filtered, and weighed.

The resin (10,000 grams) was mixed with 2-methylimidazole (250 grams) for five minutes. Benzoic anhydride (945 grams) was added, and the solution mixed an additional five minutes. After mixing, the resultant solution was poured onto a moving polyester substrate that was heated in an oven at 100° C. for 30 seconds. The gelled film was collected and placed into an acetone bath. After immersion for 24 hours, the acetone bath was exchanged for fresh acetone. The soak and exchange process was repeated six times. After the final exchange, the gelled film was removed. The acetone solvent was evaporated under a stream of air at room temperature, and subsequently dried for 2 hrs hours at 200° C. The final recovered aerogel part had open-cell structure as observed by scanning electron microscopy (SEM) performed on a Phenom Pro Scanning Electron Microscope (Phenom-World, the Netherlands), exhibited a density of 0.20 g/cm³ and porosity of >80% as measured according to ASTM D4404-10 with a Micromeritics® AutoPore V 9605 Automatic Mercury Penetrometer (Micromeritics® Instrument Corporation, U.S.A.). The final recovered film exhibited a tensile strength and elongation of 1200 psi (8.27 MPa) and 14%, respectively, at room temperature as measured according to ASTM D882-02. The film had an average pore size of 400 nm.

### Example 9

### Preparation of Air Filter Materials and Masks

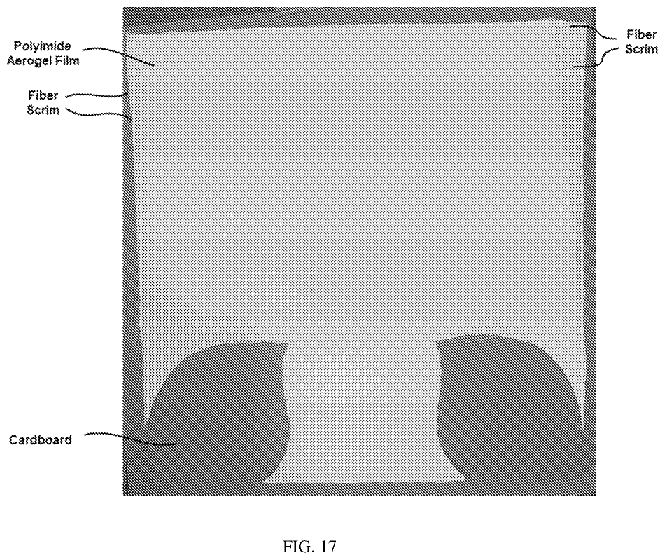

AeroZero® rolled thin film (Blueshift Materials, Inc., Spencer, Mass.) having a thickness of 165 microns was used as the air-permeable filter material. A 50 micron thick AeroZero® rolled thin film, a 125 micron thick AeroZero® rolled thin film, a 250 micron thick AeroZero® rolled thin film, and other AeroZero® rolled thin films are also available from Blueshift Materials, Inc., and can also be used in the context of the present invention. The AeroZero® rolled thin films are polyimide aerogels that can be made by the processes described throughout this specification. The thickness of the film can be modified as desired for a given air filter application or product. The rolled thin film was then adhesively attached to a Nomex® fiber scrim (DuPont, Wilmington, Del.). The Nomex® fiber scrim was Meta-Aramid Scrim 69 (obtained from Infiniti TechTex, Mumbai, India). The adhesive was a polyester-based adhesive (Bostik HM4199MV, Bostik Inc., Wauwatusa, Wis.). The following process can be used to make the air-permeable filter: (1) immerse the Nomex® fiber scrim in the polyester-based adhesive; and (2) then press together the loaded scrim with the AeroZero® rolled thin film using a mechanical roll nip set to approximately 10 pounds per square inch pressure and a feed rate of approximately 5 feet per minute. FIG. 17 provides an image of the produced Nomex® fiber scrim supported AeroZero® polyimide aerogel film, with portions cut out with scissors. The removed portions are in the general shape that can be used as the air-permeable filter material for a mask. Referring to FIG. 17, the fiber scrim supported aerogel film is positioned on a piece of cardboard, but is not affixed to the cardboard.

### Example 10

### Preparation of Air Filter Materials and Masks

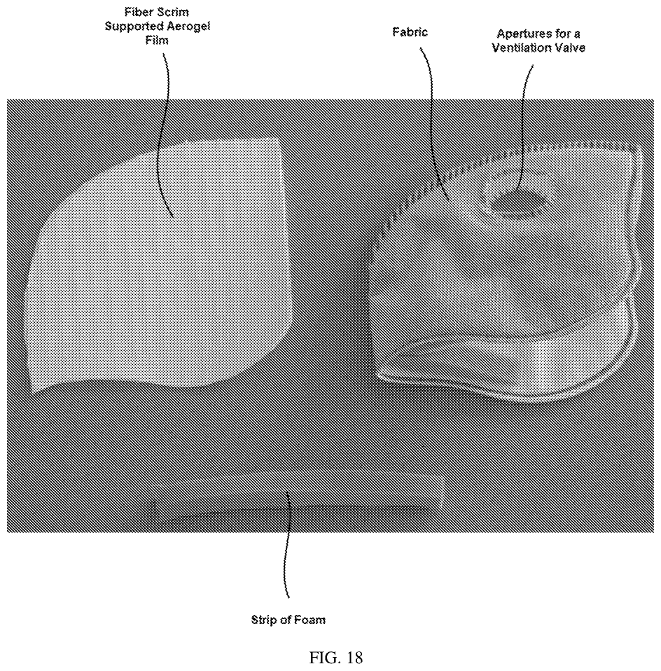

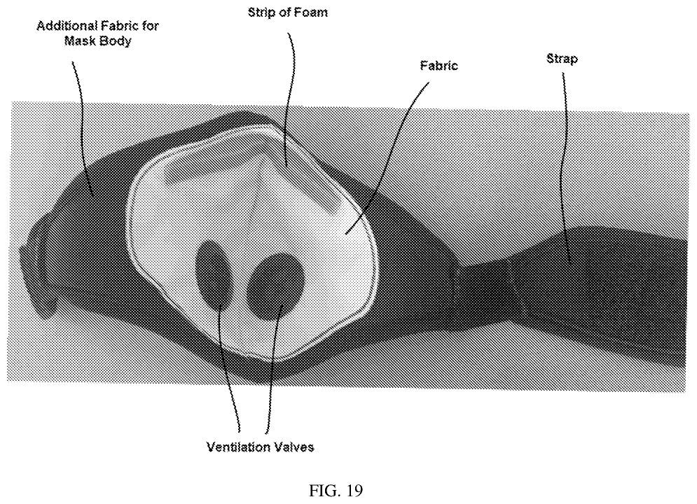

FIG. 18 illustrates parts of mask that include the removed portions of the fiber scrim supported AeroZero® polyimide aerogel film, a strip of foam that can be used to provide a cushion to a user's nose, and fabric that can be used to support the fiber scrim supported aerogel film and strip of foam.

FIG. 19 illustrates an assembled mask that also includes additional fabric for the mask body and a strap to secure the mask to a person's face. The fiber scrim supported AeroZero® polyimide aerogel film is not seen in FIG. 19, as this supported film is positioned between the fabric and the additional fabric for the mask body. The strip of foam is secured to the fabric with an adhesive. The fabric is secured to the additional fabric through threaded stitching.

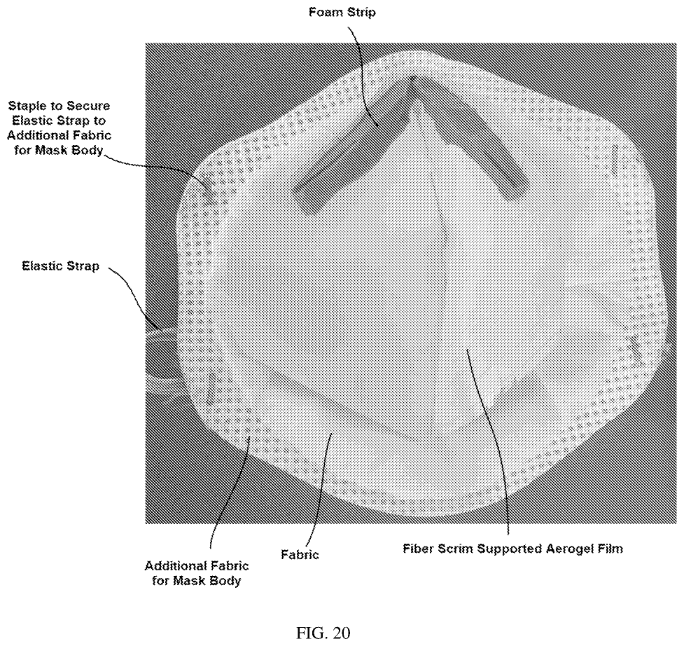

FIG. 20 is another mask that includes the foam strip, the fiber scrim supported AeroZero® polyimide aerogel film, fabric, additional fabric for the mask body, and elastic straps that can be used to secure the mask to a person's ears or to the back of a person's head. The foam strip is secured to the AeroZero® polyimide aerogel film with an adhesive. The fiber scrim supported aerogel film is secured to the fabric with an adhesive. The fabric is secured to the additional fabric with an adhesive. The elastic straps are each secured to the top and bottom portions of each side of the mask with staples, which creates two loops of elastic straps from the top and bottom left side and right side portions of the mask.

### Example 11

### Prophetic Example for Testing

### Air-Permeable Filter Materials of the Present Invention

The air-permeable filter materials of the present invention can be subjected to tests to determine the efficiency of any given parameter. Non-limiting tests that can be used in the context of the present invention include any one of, any combination of, or all of:
(1) Bacterial filtration efficiency can be measured according to ASTM F2101 as the percent of bacteria (e.g., Staphylococcus aureus) collected downstream of an air-permeable filter material of the present invention versus the bacteria provided upstream of the air-permeable filter material of the present invention in an aerosol initially comprising 1 million bacterial units at a face velocity of 12.5 cm/s and a flow rate of 30 liters per minute over an area of 40 cm².
(2) Viral filtration efficiency can be measured according to ASTM F2101 as the percent of viruses (e.g., Phi X174 bacteriophase) collected downstream of the air-permeable filter material of the present invention versus the viruses provided upstream of the air-permeable filter material of the present invention in an aerosol initially comprising 107 plaque-forming units of the virus at a flow rate of 30 liters per minute and face velocity of 12.5 cm/s over an area of 40 cm².
(3) Sub-micron particulate filtration efficiency can be measured according to ASTM F1215 (e.g., using 0.1 micron Latex spheres).
(4) Inhalation and exhalation resistance can be measured according to ASTM F2100-Standard Specification For Performance of Materials Used In Medical face Masks.

1. An air-permeable filter material that comprises a polymeric aerogel having a polymeric matrix comprising an open-cell structure.

2. The air-permeable filter material of claim 1, wherein the air-permeable filter material is comprised in a mask.

3. The air-permeable filter material of claim 2, wherein at least a portion of the mask is configured to be placed over a user's mouth and/or nose.

4. The air-permeable filter material of claim 3, wherein the mask comprises at least one layer of the air-permeable filter material and is positioned such that inhaled and/or exhaled air of the user passes through the filter material.

5. The air-permeable filter material of any one of claims 2 to 4, wherein the mask comprises at least one strap that is attached to at least two different parts of the mask, and wherein at least a portion of the strap is capable of being positioned behind a user's head or ear.

6. The air-permeable filter material of claim 5, wherein the at least one strap comprises a stretchable or elastic portion.

7. The air-permeable filter material of any one of claims 2 to 4, wherein the air-permeable filter material is comprised in a cartridge.

8. The air-permeable filter material of claim 7, wherein the cartridge is a replaceable cartridge for a cartridge-type respirator.

9. The air-permeable filter material of any one of claim 8, wherein the air-permeable filter material is in the form of a film or is in the form of particles, or a combination thereof.

10. The air-permeable filter material of claim 9, wherein the cartridge has a space, and wherein the space is at least partially filled with the film or the particles or both of the film and particles.

11. The air-permeable filter material of claim 1, wherein the material is comprised in a vent filter.

12. The air-permeable filter material of claim 11, wherein the vent filter has a first surface and an opposing second surface, wherein the shape of the first and second surfaces are square or rectangular in shape.

13. The air-permeable filter material of any one of claims 11 to 12, wherein the vent filter comprises an outer border, wherein the outer border comprises a paper material.

14. The air-permeable filter material of claim 13, wherein the paper material is cardboard or cotton.

15. The air-permeable filter material of any one of claims 1 to 4, wherein the polymeric aerogel is in the form of a film or is in particulate form or a combination thereof.

16. The air-permeable filter material of claim 15, wherein the polymeric aerogel is in the form of a film.

17. The air-permeable filter material of claim 16, wherein the film is adhesively bonded to a support, preferably a scrim.

18. The air-permeable filter material of claim 15, wherein the polymeric aerogel is in particulate form.

19. The air-permeable filter material of claim 18, wherein the polymeric aerogel in particulate form is comprised in a support material, preferably a fibrous support material.

20. The air-permeable filter material of any one of claims 1 to 4, wherein the polymeric aerogel is attached to a substrate.

21. The air-permeable filter material of claim 20, wherein the substrate comprises a paper material, a fibrous material, a metal material, a thermoplastic material, or a thermoset material.

22. The air-permeable filter material of claim 21, wherein the substrate is a fiber substrate, preferably, a woven fiber substrate, a knitted fiber substrate, non-woven fiber substrate, or a paper substrate.

23. The air-permeable filter material of claim 22, wherein the substrate is a scrim.

24. The air-permeable filter material of claim 23, wherein the polymeric aerogel is attached to the substrate with an adhesive, preferably a polyester-based adhesive.

25. The air-permeable filter material of claim 20, wherein the substrate is air permeable.

26. The air-permeable filter material of any one of claims 1 to 4, wherein the filter material is a N95, N99, or N100 qualified filter material.

27. The air-permeable filter material of any one of claims 1 to 4, wherein the filter material is a R95, R99, and R100 qualified filter material.

28. The air-permeable filter material of any one of claims 1 to 4, wherein the filter material is a P95, P99, or P100 qualified filter material.

29. The air-permeable filter material of any one of claims 1 to 4, wherein the filter material is a high efficiency (HE) qualified filter material.

30. The air-permeable filter material of any one of claims 1 to 4, wherein the filter material is capable of removing an airborne pathogen from air by passing air comprising the airborne pathogen through at least a portion of the filter material such that the airborne pathogen is retained in the polymeric aerogel and air is allowed to pass through the polymeric aerogel.

31. The air-permeable filter material of claim 30, wherein the airborne pathogen is a virus, a bacteria, a fungi, or a protozoa.

32. The air-permeable filter material of claim 31, wherein the airborne pathogen is a virus, and wherein the virus is preferably an adenovirus, alphavirus, calicivirus, coronavirus, distemper virus, Ebola virus, enterovirus, flavivirus, hepatitis virus, herpesvirus, infectious peritonitis virus, leukemia virus, Marburg virus, Norwalk virus, orthomyxovirus, papilloma virus, parainfluenza virus, the, paramyxovirus, parvovirus, pestivirus, picorna virus, pox virus, rabies virus, reovirus polypeptides, retrovirus, rotavirus, and vaccinia virus.

33. The air-permeable filter material of claim 32, wherein the airborne pathogen is a coronavirus.

34. The air-permeable filter material of claim 33, wherein the coronavirus is MERS-CoV, SARS-CoV, or SARS-CoV-2, preferably SARS-CoV-2.

35. The air-permeable filter material of claim 31, wherein the airborne pathogen is a bacteria, and wherein the bacterial is preferably Actinomyces, Bacillus, Bacteroides, Bordetella, Bartonella, Borrelia, Brucella, Campylobacter, Capnocytophaga, Chlamydia, Corynebacterium, Coxiella, Dermatophilus, Enterococcus, Ehrlichia, Escherichia, Francisella, Fusobacterium, Haemobartonella, Haemophilus, Helicobacter, Klebsiella, L-form bacteria, Leptospira, Listeria, Mycobacteria, Mycoplasma, Neisseria, Neorickettsia, Nocardia, Pasteurella, Peptococcus, Peptostreptococcus, Pneumococcus, Proteus, Pseudomonas, Rickettsia, Rochalimaea polypeptides, Salmonella, Shigella, Staphylococcus, group A streptococcus, group B streptococcus, Treponema, and Yersinia.

36. The air-permeable filter material of claim 31, wherein the airborne pathogen is a fungi, and wherein the fungi is preferably Absidia, Acremonium, Alternaria, Aspergillus, Basidiobolus, Bipolaris, Blastomyces, Candida, Coccidioides, Conidiobolus, Cryptococcus, Curvalaria, Epidermophyton, Exophiala, Geotrichum, Histoplasma, Madurella, Malassezia, Microsporum, Moniliella, Mortierella, Mucor, Paecilomyces, Penicillium, Phialemonium, Phialophora, Prototheca, Pseudallescheria, Pseudomicrodochium, Pythium, Rhinosporidium, Rhizopus, Scolecobasidium, Sporothrix, Stemphylium, Trichophyton, Trichosporon, and Xylohypha.

37. The air-permeable filter material of claim 31, wherein the airborne pathogen is a protozoa, and wherein the protozoa is preferably Babesia, Balantidium, Besnoitia, Cryptosporidium, Eimeria, Encephalitozoon, Entamoeba, Giardia, Hammondia, Hepatozoon, Isospora, Leishmania, Microsporidia, Neospora, Nosema, Pentatrichomonas, or Plasmodium.

38. The air permeable filter material of any one of claims 1 to 4, wherein the polymeric aerogel comprises micropores, mesopores, or macropores, or a combination thereof.

39. The air permeable filter material of claim 38, wherein at least 10%, 50%, 75%, 95%, or 100% of the aerogel's pore volume is made up of micropores.

40. The air permeable filter material of claim 38, wherein at least 10%, 50%, 75%, 95%, or 100% of the aerogel's pore volume is made up of mesopores.

41. The air permeable filter material of claim 38, wherein at least 10%, 50%, 75%, 95%, or 100% of the aerogel's pore volume is made up of macropores, preferably at least 90% of the aerogel's pore volume is made up of macropores.

42. The air permeable filter material of claim 41, wherein less than 90%, 80%, 70%, 60%, 50%, 40%, 30% 20%, 10% or less than 5% of the aerogel's pore volume is made up of micropores and/or mesopores, preferably less than 10% of the aerogel's pore volume is made up of micropores and/or mesopores.

43. The air permeable filter material of claim 38, wherein the polymeric matrix has an average pore size of 2 nanometers (nm) to 50 nm in diameter.

44. The air permeable filter material of claim 38, wherein the polymeric matrix has an average pore size of greater than 50 nanometers (nm) to 5000 nm in diameter, preferably 1,000 nm to 1,400 nm, or more preferably around 1,200 nm.

45. The air permeable filter material of claim 38, wherein the polymeric matrix has an average pore size of 100 nm to 800 nm, preferably 100 nm to 500 nm, more preferably from 150 nm to 400 nm, even more preferably from 200 nm to 300 nm, still more preferably from 225 nm to 275 nm.

46. The air permeable filter material of any one of claims 1 to 4, wherein the polymeric aerogel is an organic polymeric aerogel.

47. The air permeable filter material of any one of claims 1 to 4, wherein the polymeric aerogel is a polyimide aerogel.

48. The air permeable filter material of any one of claims 1 to 4, wherein the polymeric aerogel is a polyamide aerogel, a polyaramid aerogel, a polyurethane aerogel, a polyuria aerogel, or a polyester aerogel.

49. The air permeable filter material of any one of claims 1 to 4, further comprising a support film or layer at least partially penetrating the polymer matrix of the aerogel.

50. The air permeable filter material of claim 49, wherein the support film is a fiber support.

51. The air permeable filter material of claim 50, wherein the fiber support is a woven fiber support, knitted fiber support, non-woven fiber support or a paper.

52. The air permeable filter material of any one of claims 1 to 4, wherein the material is in the form of a film or layer having a thickness of 0.01 millimeters (mm) to 1000 mm thick, or from 0.025 mm to 100 mm thick, or from 0.050 mm to 10 mm thick, or from 0.100 mm to 2 mm thick, or from 0.125 mm to 1.5 mm.

53. The air permeable filter material of any one of claims 1 to 4, wherein the material is re-useable, washable, and/or capable of being disinfected and reused.

54. A method of removing airborne pathogens, the method comprising passing air comprising the airborne pathogen through at least a portion of the air permeable filter material of any one of claims 1 to 53 such that the airborne pathogen is retained in the polymeric aerogel and air is allowed to pass through the polymeric aerogel.

55. The method of claim 54, wherein the airborne pathogen is a virus, a bacteria, a fungi, or a protozoa.

56. The method of claim 55, wherein the airborne pathogen is a virus, and wherein the virus is preferably an adenovirus, alphavirus, calicivirus, coronavirus, distemper virus, Ebola virus, enterovirus, flavivirus, hepatitis virus, herpesvirus, infectious peritonitis virus, leukemia virus, Marburg virus, Norwalk virus, orthomyxovirus, papilloma virus, parainfluenza virus, the, paramyxovirus, parvovirus, pestivirus, picorna virus, pox virus, rabies virus, reovirus polypeptides, retrovirus, rotavirus, and vaccinia virus.

57. The method of claim 56, wherein the airborne pathogen is a coronavirus.

58. The method of claim 57, wherein the coronavirus is MERS-CoV, SARS-CoV, or SARS-CoV-2, preferably SARS-CoV-2.

59. The method of claim 55, wherein the airborne pathogen is a bacteria, and wherein the bacterial is preferably Actinomyces, Bacillus, Bacteroides, Bordetella, Bartonella, Borrelia, Brucella, Campylobacter, Capnocytophaga, Chlamydia, Corynebacterium, Coxiella, Dermatophilus, Enterococcus, Ehrlichia, Escherichia, Francisella, Fusobacterium, Haemobartonella, Haemophilus, Helicobacter, Klebsiella, L-form bacteria, Leptospira, Listeria, Mycobacteria, Mycoplasma, Neisseria, Neorickettsia, Nocardia, Pasteurella, Peptococcus, Peptostreptococcus, Pneumococcus, Proteus, Pseudomonas, Rickettsia, Rochalimaea polypeptides, Salmonella, Shigella, Staphylococcus, group A streptococcus, group B streptococcus, Treponema, and Yersinia.

60. The method of claim 55, wherein the airborne pathogen is a fungi, and wherein the fungi is preferably Absidia, Acremonium, Alternaria, Aspergillus, Basidiobolus, Bipolaris, Blastomyces, Candida, Coccidioides, Conidiobolus, Cryptococcus, Curvalaria, Epidermophyton, Exophiala, Geotrichum, Histoplasma, Madurella, Malassezia, Microsporum, Moniliella, Mortierella, Mucor, Paecilomyces, Penicillium, Phialemonium, Phialophora, Prototheca, Pseudallescheria, Pseudomicrodochium, Pythium, Rhinosporidium, Rhizopus, Scolecobasidium, Sporothrix, Stemphylium, Trichophyton, Trichosporon, and Xylohypha.

61. The method of claim 55, wherein the airborne pathogen is a protozoa, and wherein the protozoa is preferably Babesia, Balantidium, Besnoitia, Cryptosporidium, Eimeria, Encephalitozoon, Entamoeba, Giardia, Hammondia, Hepatozoon, Isospora, Leishmania, Microsporidia, Neospora, Nosema, Pentatrichomonas, Plasmodium.

62. A method of disinfecting the air permeable filter material of any one of claims 1 to 53, the method comprising subjecting the material to a disinfectant solution or spray capable of killing an airborne pathogen.

63. The method of claim 62, wherein the airborne pathogen is a virus, a bacteria, a fungi, or a protozoa.

64. A method of making the air permeable filter material of any one of claims 1 to 53, the method comprising:
(a) providing a monomer or a combination of monomers to a solvent to form a solution;
(b) polymerizing the monomers in the solution to form a polymer gel matrix; and
(c) subjecting the polymer gel matrix to conditions sufficient to remove liquid from the polymer gel matrix to form an aerogel having a polymeric matrix comprising an open-cell structure.

65. The method of claim 64, wherein step (b) further comprises adding a curing agent to the solution to reduce the solubility of polymers formed in the solution and to form macropores in the gel matrix, the formed macropores containing liquid from the solution.

66. The method of any one of claims 64 to 65, the method further comprising: covering or immersing a support film in the solution in step (a) or (b) such that the support film is attached to the resulting polymer gel matrix and ultimately to the produced aerogel matrix.

67. The method of any one of claims 64 to 65, further comprising casting the polymer gel matrix in step (b) onto a support such that a layer of the polymeric gel matrix is comprised on the support, wherein the aerogel in step (c) is in the form of a film.

68. The method of claim 67, further comprising crushing, grinding, or milling the film to produce aerogel particles.

69. A face mask comprising:
an air-permeable filter material that comprises a polymeric aerogel having a polymeric matrix comprising an open-cell structure, wherein at least 90% of the polymeric matrix's pore volume comprises macropores;
at least one strap that is attached to at least two different parts of the mask, wherein at least a portion of the strap is stretchable or elastic and is capable of being positioned behind a person's head or ear,
wherein at least a portion of the air-permeable filter material is configured such that inhaled and/or exhaled air of the person passes through the filter material, and
wherein the filter material is capable of removing an airborne pathogen from the air by passing the air comprising the airborne pathogen through at least a portion of the filter material such that the airborne pathogen is retained in the polymeric aerogel and the air is allowed to pass through the polymeric aerogel.

70. The face mask of claim 69, wherein the polymeric aerogel in the air-permeable filter material is in the form of a sheet or film.

71. The face mask of claim 69, wherein the polymeric aerogel in the air-permeable filter material is in particulate form.

72. The face mask of claim 71, wherein the air-permeable filter material further comprises particles of activated carbon or charcoal.

73. The face mask of any one of claims 69 to 72, wherein the airborne pathogen is a virus, preferably a coronavirus, more preferably MERS-CoV, SARS-CoV, or SARS-CoV-2, or even more preferably SARS-CoV-2.

74. The face mask of claim 73, wherein the polymer aerogel is a polyimide aerogel.

75. The face mask of any one of claims 69 to 72, wherein the polymeric matrix has an average pore size of greater than 50 nanometers (nm) to 5000 nm in diameter, preferably 1,000 nm to 1,400 nm in diameter or 100 nm to 800 nm in diameter.
